(19) United States
(12) Patent Application Publication
(10) Pub. No.: US 2005/0203059 A1
(43) Pub. Date: Sep. 15, 2005

(54) Methods and compositions for treatment of ischemia

(76) Inventors: Stephen D. Harrison, Emeryville, CA (US); Allan S. Wagman, Emeryville, CA (US); Kathleen A. Martin, Emeryville, CA (US)

Correspondence Address: Chiron Corporation;Intellectual Property R 338, PO Box 8097, Emeryville, CA, 94662-8097, US

(21) Appl. No.: 10/506,570

(22) PCT Filed: Mar. 3, 2003
(86) PCT No.: PCT/US03/06742

§ 371 Date: Apr. 29, 2005

Related U.S. Application Data
(60) Provisional application No. 60/361,417, filed on Mar. 1, 2002.

(51) Int. Cl. A61K 31/727; A61K 31/5377; A61K 31/496; A61K 31/4439; A61K 31/366

(52) U.S. Cl. 514/56; 514/341; 514/332; 514/235.2; 514/253.01; 514/457; 514/318

(57) ABSTRACT

Methods and compositions are provided for the prophylaxis or inhibition of cerebral ischemic injury by administration of an inhibitor of glycogen synthase kinase 3 (GSK3) activity, either alone or in combination with at least one additional agent for the treatment of ischemic stroke.

### FIELD OF THE INVENTION

This invention relates to methods of treatment of ischemia, especially cerebral ischemia, by administering to a human or animal subject in need of treatment an amount of a glycogen synthase kinase 3 (GSK3) inhibitor effective to reduce ischemic damage to the subject in the event of a traumatic event, such as a stroke. The invention further relates to compositions for the prophylaxis or inhibition of ischemic injury comprising a glycogen synthase kinase 3 (GSK3) inhibitor and at least one additional agent for the treatment of ischemic damage.

### BACKGROUND OF THE INVENTION

Stroke is the leading cause of disability and a major cause of death in this country. To date, proven treatments for this devastating illness are few, although many studies both at the laboratory and clinical levels are in progress. Recent research in the area of neurodegeneration has shown that β-catenin, a protein involved in protein-protein interactions, is involved in transcriptional regulation after signaling via the Wnt pathway (T. C. Dale, “Signal transduction by the Wnt family of ligands,” Biochemical Journal, 329:209-23 (1998)). β-catenin binds to the DNA-binding protein, TCF (T-cell factor) which results in transcription of various target genes. These pathways are thought to be important in cell proliferation. β-catenin is inhibited by phosphorylation via glycogen synthase kinase-3β (GSK-3). Furthermore, GSK-3 may also phosphorlyate tau protein when bound to mutant presenilin 1 (A. Takashima et al., “Presenilin 1 associates with glycogen synthase kinase-3beta and its substrate tau,” Proceedings of the National Academy of Sciences of the United States of America 95:9637-41 (1998)) and destabilize β-catenin leading to apoptosis (Z. Zhang et al., “Destabilization of beta-catenin by mutations in presenilin-1 potentiates neuronal apoptosis,” Nature 395:698-702 (1998)). Overexpression of GSK-3 induced apoptosis of both Rat-1 and PC12 cells (M. Pap et al., “Role of glycogen synthase kinase-3 in the phosphatidylinositol 3-Kinase/Akt cell survival pathway,” Journal of Biological Chemistry, 273:19929-32 (1998)). Following cerebral ischemia, it is now known that apoptosis (programmed cell death) occurs to a certain extent, and that appropriate biochemical or genetic alteration of this process leads to neuroprotection. For example, it has been shown that overexpression of the anti-apoptotic protein, Bcl-2 protected striatal neurons from transient focal ischemia (M. S. Lawrence et al., “Overexpression of Bcl-2 with herpes simplex virus vectors protects CNS neurons against neurological insults in vitro and in vivo,” J Neurosci 16:486-96 (1996); M. A. Yenari et al., “Herpes simplex viral vectors expressing Bcl-2 are neuroprotective against focal cerebral ischemia” In J. Krieglstein (Ed.), Pharmacology of Cerebral Ischemia, Wissenschaftliche Verlagsgesellschaft mbH, Stuttgart, 1996, pp. 537-543) even when Bcl-2 overexpression was applied after ischemia onset (M. S. Lawrence et al., “Herpes simplex viral vectors expressing Bcl-2 are neuroprotective when delivered after a stroke,” J Cereb Blood Flow Metab 17:740-4 (1997); M. A. Yenari et al., “Herpes simplex viral vectors expressing Bcl-2 are neuroprotective against focal cerebral ischemia” In J. Krieglstein (Ed.), Pharmacology of Cerebral Ischemia, Wissenschaftliche Verlagsgesellschaft mbH, Stuttgart, 1996, pp. 537-543). Pharmacologic inhibition with caspase inhibitors has also been shown to be neuroprotective against experimental stroke (F. J. Gottron et al., “Caspase inhibition selectively reduces the apoptotic component of oxygen-glucose deprivation-induced cortical neuronal cell death,” Molecular and Cellular Neurosciences 9:159-69 (1997); T. Himi et al., “A caspase inhibitor blocks ischaemia-induced delayed neuronal death in the gerbil,” European Journal of Neuroscience 10:777-81 (1998); S. Namura et al., “Activation and cleavage of caspase-3 in apoptosis induced by experimental cerebral ischemia,” Journal of Neuroscience 18:3659-68 (1998). Additionally, it has been shown that caspase based inhibitors can be protective when administered post reperfusion (Mouw et al., “Caspase-9 inhibition after focal cerebral ischemia improves outcome following reversible focal ischemia,” Metab Brain Dis 17(3):143-51 (2002)).

Glycogen synthase kinase 3 (GSK3) is a serine/threonine kinase for which two isoforms, α and β, have been identified. Woodgett, Trends Biochem. Sci., 16:177-81 (1991). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such glucose transport. Subsequently, it has been shown that GSK3 activity is also inactivated by other growth factors that, like insulin, signal through receptor tyrosine kinases (RTKs). Examples of such signaling molecules include IGF-1 and EGF. Saito et al., Biochem. J., 303:27-31 (1994); Welsh et al., Biochem. J. 294:625-29 (1993); and Cross et al., Biochem. J., 303:21-26 (1994).

Agents that inhibit GSK3 activity are useful in the treatment of disorders that are mediated by GSK3 activity. In addition, inhibition of GSK3 mimics the activation of growth factor signaling pathways and consequently GSK3 inhibitors are useful in the treatment of diseases in which such pathways are insufficiently active. Examples of diseases that can be treated with GSK3 inhibitors are described below.

Diabetes.

Type 2 diabetes is an increasingly prevalent disease of aging. It is initially characterized by decreased sensitivity to insulin and a compensatory elevation in circulating insulin concentrations, the latter of which is required to maintain normal blood glucose levels. Increased insulin levels are caused by increased secretion from the pancreatic beta cells, and the resulting hyperinsulinemia is associated with cardiovascular complications of diabetes. As insulin resistance worsens, the demand on the pancreatic beta cells steadily increases until the pancreas can no longer provide adequate levels of insulin, resulting in elevated levels of glucose in the blood. Ultimately, overt hyperglycemia and hyperlipidemia occur, leading to the devastating long-term complications associated with diabetes, including cardiovascular disease, renal failure and blindness. The exact mechanism(s) causing type 2 diabetes are unknown, but result in impaired glucose transport into skeletal muscle and increased hepatic glucose production, in addition to inadequate insulin response. Dietary modifications are often ineffective, therefore the majority of patients ultimately require pharmaceutical intervention in an effort to prevent and/or slow the progression of the complications of the disease. Many patients can be treated with one or more of the many oral anti-diabetic agents available, including sulfonylureas, to increase insulin secretion. Examples of sulfonylurea drugs include metformin for suppression of hepatic glucose production, and troglitazone, an insulin-sensitizing medication. Despite the utility of these agents, 30-40% of diabetics are not adequately controlled using these medications and require subcutaneous insulin injections. Additionally, each of these therapies has associated side effects. For example, sulfonylureas can cause hypoglycemia and troglitazone can cause severe hepatoxicity. Presently, there is a need for new and improved drugs for the treatment of prediabetic and diabetic patients.

As described above, GSK3 inhibition stimulates insulin-dependent processes and is consequently useful in the treatment of type 2 diabetes. Recent data obtained using lithium salts provides evidence for this notion. The lithium ion has recently been reported to inhibit GSK3 activity. Klein et al., PNAS 93:8455-9 (1996). Since 1924, lithium has been reported to have antidiabetic effects including the ability to reduce plasma glucose levels, increase glycogen uptake, potentiate insulin, up-regulate glucose synthase activity and to stimulate glycogen synthesis in skin, muscle and fat cells. However, lithium has not been widely accepted for use in the inhibition of GSK3 activity, possibly because of its documented effects on molecular targets other than GSK3. The purine analog 5-iodotubercidin, also a GSK3 inhibitor, likewise stimulates glycogen synthesis and antagonizes inactivation of glycogen synthase by glucagon and vasopressin in rat liver cells. Fluckiger-Isler et al., Biochem J 292:85-91 (1993); and Massillon et al., Biochem J 299:123-8 (1994). However, this compound has also been shown to inhibit other serine/threonine and tyrosine kinases. Massillon et al., Biochem J 299:123-8 (1994).

Alzheimer's Disease.

GSK3 is also involved in biological pathways relating to Alzheimer's disease (AD). The characteristic pathological features of AD are extracellular plaques of an abnormally processed form of the amyloid precursor protein (APP), so called β-amyloid peptide (β-AP) and the development of intracellular neurofibrillary tangles containing paired helical filaments (PHF) that consist largely of hyperphosphorylated tau protein. GSK3 is one of a number of kinases that have been found to phosphorylate tau protein in vitro on the abnormal sites characteristic of PHF tau, and is the only kinase also demonstrated to do this in living cells and in animals. Lovestone et al., Current Biology 4:1077-86 (1994); and Brownlees et al., Neuroreport 8: 3251-3255 (1997). Furthermore, the GSK3 kinase inhibitor, LiCl, blocks tau hyperphosphorylation in cells. Stambolic et al., Current Biology 6:1664-8 (1996). Thus GSK3 activity may contribute to the generation of neurofibrillary tangles and consequently to disease progression. Recently it has been shown that GSK3B associates with another key protein in AD pathogenesis, presenillin 1 (PS1). Takashima et., PNAS 95:9637-9641 (1998). Mutations in the PS1 gene lead to increased production of β-AP, but the authors also demonstrate that the mutant PS1 proteins bind more tightly to GSK3B and potentiate the phosphorylation of tau, which is bound to the same region of PS1.

Interestingly it has also been shown that another GSK3 substrate, β-catenin, binds to PS1. Zhong et al., Nature 395:698-702 (1998). Cytosolic β-catenin is targeted for degradation upon phosphorylation by GSK3 and reduced β-catenin activity is associated with increased sensitivity of neuronal cells to β-AP induced neuronal apoptosis. Consequently, increased association of GSK3β with mutant PS1 may account for the reduced levels of β-catenin that have been observed in the brains of PS1-mutant AD patients and to the disease related increase in neuronal cell-death. Consistent with these observations, it has been shown that injection of GSK3 antisense but not sense, blocks the pathological effects of β-AP on neurons in vitro, resulting in a 24 hr delay in the onset of cell death and increased cell survival at 1 hr from 12 to 35%. Takashima et al., PNAS 90:7789-93. (1993). In these latter studies, the effects on cell-death are preceded (within 3-6 hours of β-AP administration) by a doubling of intracellular GSK3 activity, suggesting that in addition to genetic mechanisms that increase the proximity of GSK3 to its substrates, β-AP may actually increase GSK3 activity. Further evidence for a role for GSK3 in AD is provided by the observation that the protein expression level (but, in this case, not specific activity) of GSK3 is increased by 50% in postsynaptosomal supernatants of AD vs. normal brain tissue. Pei et al., J Neuropathol Exp 56:70-78 (1997). Thus, it is believed that specific inhibitors of GSK3 will act to slow the progression of Alzheimer's disease.

Other CNS Disorders

In addition to the effects of lithium described above, there is a long history of the use of lithium to treat bipolar disorder (manic depressive syndrome). This clinical response to lithium may reflect an involvement of GSK3 activity in the etiology of bipolar disorder, in which case GSK3 inhibitors could be relevant to that indication. In support of this notion it was recently shown that valproate, another drug commonly used in the treatment of bipolar disorder, is also a GSK3 inhibitor. Chen et al., J. Neurochemistry 72:1327-1330 (1999). One mechanism by which lithium and other GSK3 inhibitors may act to treat bipolar disorder is to increase the survival of neurons subjected to aberrantly high levels of excitation induced by the neurotransmitter, glutamate. Nonaka et al., PNAS 95: 2642-2647 (1998). Glutamate-induced neuronal excitotoxicity is also believed to be a major cause of neurodegeneration associated with acute damage, such as in cerebral ischemia, traumatic brain injury and bacterial infection. Furthermore it is believed that excessive glutamate signaling is a factor in the chronic neuronal damage seen in diseases such as Alzheimer's, Huntingdon's, Parkinson's, AIDS associated dementia, amyotrophic lateral sclerosis (AML) and multiple sclerosis (MS). Thomas, J. Am. Geriatr. Soc. 43: 1279-89 (1995). Consequently GSK3 inhibitors are believed to be a useful treatment in these and other neurodegenerative disorders.

Immune Potentiation

GSK3 phosphorylates transcription factor NF-AT and promotes its export from the nucleus, in opposition to the effect of calcineurin (Beals et al., Science 275:1930-33 (1997).) Thus, GSK3 blocks early immune response gene activation via NF-AT, and GSK3 inhibitors may tend to permit or prolong activation of immune responses. Thus GSK3 inhibitors are believed to prolong and potentiate the immunostimulatory effects of certain cytokines, and such an effect may enhance the potential of those cytokines for tumor immunotherapy or indeed for immunotherapy in general.

Other Disorders

Lithium also has other biological effects. It is a potent stimulator of hematopoiesis, both in vitro and in vivo (Hammond et al., Blood 55: 26-28 (1980)). In dogs, lithium carbonate eliminated recurrent neutropenia and normalized other blood cell counts (Doukas et al. Exp Hematol 14: 215-221 (1986)). If these effects of lithium are mediated through the inhibition of GSK3, GSK3 inhibitors may have even broader applications.

GSK3 Inhibitors

Various GSK3 inhibitory compounds and methods of their synthesis and use are disclosed in U.S. and international patent application Publication Nos. 20020156087, WO0220495 and WO9965897 (pyrimidine and pyridine based compounds); 20030008866, 20010044436 and WO0144246 (bicyclic based compounds); 20010034051 (pyrazine based compounds); and WO9816528 (purine based compounds). Furthermore, additional GSK3 inhibitory compounds useful within the context of this invention include those disclosed in WO0222598 (quinolinone based compounds). The entire disclosure of these U.S. and international publications is incorporated herein by this reference.

It has now been discovered that GSK-3β is involved in potentiating ischemic injury, and that its inhibition with GSK3 inhibitors, such as those disclosed in the above reference international patent applications, can reduce ischemic injury, such as ischemic injury resulting from stroke.

### SUMMARY OF THE INVENTION

In accordance with the present invention, methods are provided for the prophylaxis or inhibition of ischemic injury and/or for treating cerebrovascular ischemic disorders in a human or animal subject in need of treatment by administration to the subject within 24 hours of the onset of the ischemic stroke event an amount of an inhibitor of glycogen synthase kinase 3 (GSK3) activity effective to reduce or prevent ischemic injury in the subject. In one aspect of the invention, the subject is a human or animal subject suffering from a cerebrovascular ischemic disorder.

In other aspects, the present invention provides methods for treating cerebrovascular ischemic disorders in a human or animal subject in need of such treatment comprising administering to said subject an amount of a glycogen synthase kinase 3 (GSK3) inhibitor effective to reduce or prevent ischemic injury in the subject in combination with at least one additional agent for the treatment of ischemic stroke.

In yet other aspects, the present invention provides therapeutic compositions comprising at least one GSK3 inhibitor compound in combination with one or more additional agents for the treatment of ischemic stroke, as are commonly employed in stroke therapy.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a graphic representation showing the percentage of rat hippocampi cell survival versus CHO cell EC₅₀ values of representative GSK3 inhibitor compounds as described in Example 2.

FIG. 2 is a graphic representation showing the reduction in ischemic area in rat brain tissue when treated by a GSK3 inhibitor, CT 99025, as compared to control in the rodent model of transient middle cerebral artery occlusion (MCAO) as described in Example 5.

### DETAILED DESCRIPTION OF THE PREFERRED EMBODIMENT

In accordance with the present invention, methods are provided for the prophylaxis or inhibition of ischemic injury in a human or animal subject in need of treatment by administration to the subject an inhibitor of glycogen synthase kinase 3 (GSK3) activity, either in vitro or in vivo.

In one aspect of the invention, the subject is a human or animal subject suffering from a cerebrovascular ischemic disorder. Cerebrovascular ischemic disorders are generally caused by insufficient cerebral circulation, and include transient ischemic attacks (TIAs) and ischemic stroke. Congenital anomalies and atherosclerosis can interrupt intracranial or extracranial arterial blood flow and impair collateral flow, causing brain ischemia and consequent symptoms of neurological dysfunction. If the blood supply is promptly restored, brain tissues may recover and symptoms may disappear, but if ischemia lasts longer than 1 hour, infarction and permanent neurological damage commonly result. Thrombi or emboli due to atherosclerosis or other disorders (e.g., arteritis, rheumatic heart disease) commonly cause ischemic arterial obstruction. Atheromas, which underlie most thrombi, may affect any major cerebral artery. Large atheromas usually affect the common carotid and vertebral arteries at their origins, but the cervical bifurcation of the common carotid artery is a common site giving rise to emboli that cause strokes. Intracranial thrombosis may occur in one of the large arteries at the base of the brain, in a deep perforating artery, or in a small cortical branch, but the main trunk of the middle cerebral artery and its branches are the most common sites. Whether ischemia and/or infarction occurs depends on the efficiency of collateral circulation; e.g., concomitant stenosis of both vertebral arteries can compromise collateral circulation and intensify the effects of carotid lesions.

Most TIAs are caused by cerebral emboli from ulcerated atherosclerotic plaques in the carotid or vertebral arteries in the neck or from mural thrombi in a diseased heart. Some TIAs are caused by a brief reduction in blood flow through stenosed arteries. TIAs typically begin suddenly, last 2 to 30 minutes or more, and then abate without persistent neurological abnormalities. When TIAs last for several hours, patients may experience infarcts, even without persistent neurological abnormalities. Symptoms may be identical to those of stroke but are transient. Confusion, vertigo, binocular blindness, diplopia, and unilateral or, more often, bilateral weakness or paresthesias of the extremities may be present. Slurred speech (dysarthria) may occur with carotid or vertebrobasilar involvement. Patients with TIAs are at a markedly increased risk of stroke.

Ischemic stroke may initially occur as a stroke in evolution manifested by a deficiency in neurological performance that worsen over 24 to 48 hours, or as a completed stroke or brain infarct manifested by stable neurological injury. Typically, strokes are a result of arteriosclerotic or hypertensive stenosis, thrombosis, or embolism. The onset of stroke is commonly rapid. In evolving stroke, neurological dysfunction (often beginning in one arm, then spreading progressively) commonly extends painlessly over several hours to a day or two. Progression may occur in a stepwise manner, interrupted by periods of stability, or may be continuous. In acute completed stroke, symptoms develop rapidly, typically becoming maximal within a few minutes. An evolving stroke may become a completed stroke. During the first 48 to 72 hours of an evolving stroke or of a large completed stroke, deficiency in neurological performance may worsen and consciousness may become clouded because of cerebral edema.

During the first days of an ischemic stroke, neither progression nor outcome can be predicted. About 20% of patients die in the hospital; the mortality rate increases with age. The extent of neurological recovery depends on factors such as the patient's age and general state of health and on the site and size of the infarct. Impaired consciousness, mental deterioration, aphasia, or severe brain stem signs typically suggest a poor prognosis. Complete recovery is uncommon, but the sooner improvement begins, the better the likely outcome for the patient. About 50% of patients with moderate or severe hemiplegia and most with milder deficits recover functionally by the time of discharge and can eventually care for their basic needs, have a clear sensorium, and can walk adequately, although use of an affected limb may be limited. Any deficit remaining after 6 months is likely to be permanent, although some patients continue to improve slowly. Cerebral infarction recurs relatively often, and each recurrence is likely to add to the neurological disability.

Thus, in one aspect, the present invention provides methods for treating cerebrovascular ischemic disorders in a human or animal subject in need of such treatment comprising administering to said subject an amount of a glycogen synthase kinase 3 (GSK3) inhibitor effective to reduce or prevent ischemic injury in the subject. A reduction of prevention of ischemic injury can be determined by a reduction or absence of anticipated symptoms conventionally associated with ischemic brain damage, such as, for example, a deficiency in neurological performance, impaired consciousness, mental deterioration, confusion, vertigo, binocular blindness, diplopia (double vision), aphasia (loss or impairment of the power to use or comprehend words), dysarthria (slurred speech), hemiplegia (total or partial paralysis of one side of the body), and/or a reduction in control of motor skills. Due to the evolving nature of ischemic damage due to ischemic stroke, initiation of treatment of the patient in accordance with the invention is preferably initiated within 24 hours, more preferably within 8 hours and most preferably within 2 hours of the onset of the ischemic stroke event. Once initiated, treatment of the patient in accordance with the invention may be continued, intermittently or continuously, for periods of at least 8 hours, more preferably for at least 24 hours and most preferably for at least 48 hours or longer, until the conditions resulting in potential ischemic injury have been abated.

In other aspects, the present invention provides methods for treating cerebrovascular ischemic disorders in a human or animal subject in need of such treatment comprising administering to said subject an amount of a glycogen synthase kinase 3 (GSK3) inhibitor effective to reduce or prevent ischemic injury in the subject in combination with at least one additional agent for the treatment of ischemic stroke. Useful additional agents include agents commonly or experimentally used in connection with stroke therapy, such as, for example, thrombolytic and/or fibrinolytic agents that help reestablish cerebral circulation by dissolving (lysing) the clots which obstruct blood flow, neuroprotective agents that work to minimize the effects of the ischemic cascade, anticoagulants and antiplatelet agents. Representative thrombolytic agents include, for example, alteplase (tissue plasminogen activator (t-PA)), an enzyme found naturally in the body which converts, or activates, plasminogen into the enzyme plasmin to dissolve a blood clot; anistreplase; reteplase; urokinase; and streptokinase. Representative neuroprotective agents include, for example, caspase inhibitors that selectively reduce the apoptotic component of oxygen-glucose deprivation-induced cortical neuronal cell death, glutamate antagonists that interfere with the progression of glutamate into the neurons, calcium antagonists that block the intracellular build-up of calcium through electrically-operated channels, opiate antagonists that interfere with the ischemic cascade by working on the opiate receptors which are overstimulated by the chemical cascade that occurs during cellular death, GABA-A agonists such as Clomethiazole (Astra) that activate the GABA-A receptor and thereby counteract the electrical activity of certain glutamate receptors, calpain inhibitors, NMDA receptor antagonists such as C-101,606 (Pfizer), K⁺ channel modulators such as BMS-204352 (Bristol-Myers Squibb), PDH kinase inhibitors, and antioxidants that scavenge free radicals generated by the ischemic cascade. In addition, the methods and compounds of the invention may be used in combination with oxygenated fluorocarbon nutrient emulsion (OFNE) therapy and/or neuroperfusion in which oxygen-rich blood is rerouted through the brain to reduce damage from an ischemic stroke. Representative anticoagulants include, for example, heparin, warfarin, dalteparin, danaparoid, enoxaparin, tinzaparin, 4-hydroxycoumarin, dicumarol, phenprocoumon, acenocoumarol, anisindone, lepirudin and indane-1,3-dione. Representative antiplatelet agents include, for example, aspirin, clopidogrel, ticlopidine, abciximab, eptifibatide, tirofiban and dipyridamole.

In this aspect of the invention, the glycogen synthase kinase 3 (GSK3) inhibitor is preferably administered to the subject prior to and/or concurrently with administration of the at least one additional agent. Administration of the glycogen synthase kinase 3 (GSK3) inhibitor prior to and/or concurrently with administration of the at least one additional agent is particularly preferred when the additional agent is a thrombolytic and/or fibrinolytic agent in order to protect the patient from reperfusion enhanced ischemic cell damage. Thus, the glycogen synthase kinase 3 (GSK3) inhibitor may be administered prior to the additional agent, concurrently with the additional agent or intermittently with the additional, as may be desired to obtain optimal protection from ischemic cell damage.

Inhibitors of GSK3 activity useful in the practice of the invention include compounds known to exhibit GSK3 inhibitory activity. In the case of cerebral ischemia, the GSK3 inhibitors are preferably selected for their ability to penetrate the blood/brain barrier to permit accumulation of effective levels of the inhibitors at a site of ischemic injury in the brain. Drug delivery to the brain continues to be a vexing problem. Systemic administration is often ineffective because the blood/brain barrier (BBB) excludes the transit of most compounds from the vasculature into nervous system tissue. Because the BBB restricts the entry of many potentially therapeutic molecules, various strategies have been devised for brain drug delivery. One direct means of targeting drugs to the brain is to deliver the drugs locally using implantable pumps, biodegradable polymers or genetically engineered cells. While these approaches each have some merit, combinations of these technologies are likely to yield the maximum opportunities for brain targeting. Convergent advances in developmental biology, human genomics, tissue and organ reconstruction, drug delivery and materials engineering have all advanced to a remarkably sophisticated level. The intersection of these disciplines is likely to lead to a revolution in localized brain drug delivery. Other approaches which do not depend on invasive neurosurgical procedures include pharmacological modulation of the BBB based on stimulating endogenous receptors constitutively expressed on the brain endothelial cells of the BBB; and receptor-mediated transport across the BBB, utilizing receptor systems that naturally exist and serve to carry molecules into the brain.

Various properties are applicable to a GSK3 inhibitor's ability to penetrate the blood/brain barrier, including for example, low molecular weight, relatively high lipophilicity and a relatively low polar surface area.

Thus, presently preferred GSK3 inhibitor compounds efficacious in the prophylaxis or treatment of ischemic injury in accordance with the invention comprise small molecule GSK3 inhibitor compounds having a relatively low molecular weight. In one aspect, the GSK3 inhibitor compounds used in the practice of the invention will have a molecular weight below about 800, more preferably below about 500 and even more preferably below 400 MW.

In another aspect, the GSK3 inhibitor compounds used in the practice of the invention will preferably exhibit a log P in the range of about 0 to 8, more preferably in the range of about 1 to 6, and even more preferably in the range of about 2 to 5, as determined by the software program PrologP 5.1 (CompuDrug International, Inc. 705 Grandview Drive, South San Francisco, Calif. 94080 USA), as described in detail in Example 3. LogP is the logarithm of the partition coefficient of the neutral form of a compound between octanol and water, and is a physico-chemical parameter that has a correlation with absorption of small molecules into physiological membranes.

In yet other aspects of the invention, compounds that are highly membrane bound often get caught at the cerebral capillary endothelium. It is therefore presently preferred that the GSK-3 inhibitor compounds used in the practice of the invention do not have permanent positive charges like choline, hydrophobic side chains, or phosphate acids. Further preferred embodiments of the instant invention include GSK-3 inhibitor compounds having a polar surface area in the range of about 90 to about 200 Å² and 5 or fewer H-bonding groups. For a discussion of characteristics of compounds capable of penetrating the blood brain barrier the following references are cited and fully incorporated by reference as if included herein: 1) Mertsch et al., “Blood-brain barrier penetration and drug development from an industrial point of view,” Current Medicinal Chemistry: Central Nervous System Agents 2(3): 187-201 (2002); 2) Filmore, “Breeching the blood: researchers are developing tactics to deliver therapeutics to the well-guarded “ruler of the organs,” Modern Drug Discovery 5(6): 22-24, 27 (2002); 3) Emerich, “Recent efforts to overcome the blood-brain barrier for drug delivery,” Expert Opinion on Therapeutic Patents 10(3):279-287 (2000); Clark et al., J. Pharm. Sci. 88:815 et seq. (1999).

Although GSK3 inhibitors that cross the blood-brain barrier are presently preferred for systemic administration, GSK3 inhibitors that do not readily cross the blood-brain barrier following systemic absorption are useful for the treatment of ischemia when administered intrathecally or intracerebrally.

In some embodiments, representative GSK3 inhibitor compounds useful in the practice of the invention include compounds capable of crossing the blood/brain barrier and having the structure of the following formula (I): 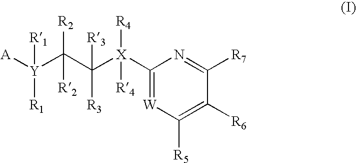
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylamino, aralkylamino, heteroaralkylamino, arylamino, heteroarylamino cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteraralkylcarbonyl, alkylcarbonyloxy, arylcarbonyloxy, aralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonyl amino, arylaminocarbonylamino, aralkylcarbonylamino, heteroaralkylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido;
and the pharmaceutically acceptable salts thereof.

The synthesis and GSK3 inhibitory activity of compounds of formula (I) is disclosed in international patent application publication No. WO9965897, published Dec. 23, 1999, the disclosure of which is incorporated herein by reference.

In some embodiments of the invention, at least one of X and Y in formula (I) is nitrogen. Representative compounds of this group include those compounds in which one of X and Y is nitrogen and the other of X and Y is oxygen or optionally substituted carbon. Preferably, both X and Y are nitrogen.

The constituent A in formula (I) can be an aromatic ring having from 3 to 10 carbon ring atoms and optionally 1 or more ring heteroatoms. Thus, in one embodiment, A can be optionally substituted carbocyclic aryl. Alternatively, A is optionally substituted heteroaryl, such as, for example, substituted or unsubstituted pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, which may substituted with at least one and not more than 3 substitution groups. Representative substitution groups can be independently selected from the group consisting of, for example, nitro, amino, cyano, halo, thioamido, amidino, oxamidino, alkoxyamidino, imidino, guanidino, sulfonamido, carboxyl, formyl, loweralkyl, haloloweralkyl, loweralkoxy, haloloweralkoxy, loweralkoxyalkyl, loweralkylaminoloweralkoxy, loweralkylcarbonyl, loweraralkylcarbonyl, lowerheteroaralkylcarbonyl, alkylthio, aminoalkyl and cyanoalkyl.

In some embodiments of the invention, A has the formula: 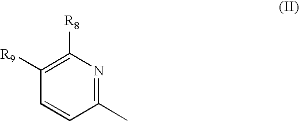
wherein R₈ and R₉ are independently selected from the group consisting of hydrogen, nitro, amino, cyano, halo, thioamido, amidino, oxamidino, alkoxyamidino, imidino, guanidinyl, sulfonamido, carboxyl, formyl, loweralkyl, haloloweralkyl, loweralkoxy, haloloweralkoxy, loweralkoxyalkyl, loweralkylaminoloweralkoxy, loweralkylcarbonyl, loweraralkylcarbonyl, lowerheteroaralkylcarbonyl, alkylthio, aryl and, aralkyl. Most preferably, A is selected from the group consisting of nitropyridyl, aminonitropyridyl, cyanopyridyl, cyanothiazolyl, aminocyanopyridyl, trifluoromethylpyridyl, methoxypyridyl, methoxynitropyridyl, methoxycyanopyridyl and nitrothiazolyl.

In other embodiments of the invention at least one of R₁, R₂, R₃ and R₄ may be hydrogen, or unsubstituted or substituted loweralkyl selected from the group consisting of haloloweralkyl, heterocycloaminoalkyl, and loweralkylaminoloweralkyl; or loweralkylaminoloweralkyl. Presently preferred embodiments of the invention include compounds wherein R₁, R₂, and R₃ are hydrogen and R₄ is selected from the group consisting of hydrogen, methyl, ethyl, aminoethyl, dimethylaminoethyl, pyridylethyl, piperidinyl, pyrrolidinylethyl, piperazinylethyl and morpholinylethyl.

Other embodiments of the invention include compounds of formula (I) wherein at least one of R₅ and R₇ is selected from the group consisting of substituted and unsubstituted aryl, heteroaryl and biaryl. In some embodiments, at least one of R₅ and R₇ is a substituted or unsubstituted moiety of the formula: 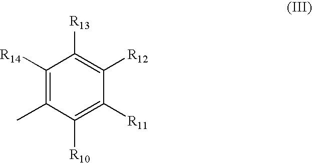
wherein R₁₀, R₁₁, R₁₂, R₁₃, and R₁₄ are independently selected from the group consisting of hydrogen, nitro, amino, cyano, halo, thioamido, carboxyl, hydroxy, and optionally substituted loweralkyl, loweralkoxy, loweralkoxyalkyl, haloloweralkyl, haloloweralkoxy, aminoalkyl, alkylamino, alkylthio, alkylcarbonylamino, aralkylcarbonylamino, heteroaralkylcarbonylamino, arylcarbonylamino, heteroarylcarbonylamino aminocarbonyl, loweralkylaminocarbonyl, aminoaralkyl, loweralkylaminoalkyl, aryl, heteroaryl, cycloheteroalkyl, aralkyl, alkylcarbonyloxy, arylcarbonyloxy, aralkylcarbonyloxy, arylcarbonyloxyalkyl, alkylcarbonyloxyalkyl, heteroarylcarbonyloxyalkyl, aralkycarbonyloxyalkyl, and heteroaralkcarbonyloxyalkyl. Presently particularly preferred compounds are obtained wherein R₁₀, R₁₁, R₁₃, and R₁₄ are hydrogen and R₁₂ is selected from the group consisting of halo, loweralkyl, hydroxy, loweralkoxy, haloloweralkyl, aminocarbonyl, alkylaminocarbonyl and cyano; R₁₁, R₁₃, and R₁₄ are hydrogen and R₁₀ and R₁₂ are independently selected from the group consisting of halo, loweralkyl, hydroxy, loweralkoxy, haloloweralkyl and cyano; R₁₀, R₁₁, R₁₃, and R₁₄ are hydrogen and R₁₂ is heteroaryl; R₁₀, R₁₁, R₁₃, and R₁₄ are hydrogen and R₁₂ is a heterocycloalkyl; and wherein at least one of R₁₀, R₁₁, R₁₂, R₁₃, and R₁₄ are halo and the remainder of R₁₀, R₁₁, R₁₂, R₁₃, and R₁₄ are hydrogen. Preferably, at least one of R₅ and R₇ is selected from the group consisting of dichlorophenyl, difluorophenyl, trifluoromethylphenyl, chlorofluorophenyl, bromochlorophenyl, ethylphenyl, methylchlorophenyl, imidazolylphenyl, cyanophenyl, morphlinophenyl and cyanochlorophenyl.

In other representative embodiments of the invention, R₆ in formula (I) may be substituted alkyl, such as, for example, aralkyl, hydroxyalkyl, aminoalkyl, aminoaralkyl, carbonylaminoalkyl, alkylcarbonylaminoalkyl, arylcarbonylaminoalkyl, aralkylcarbonylaminoalkyl, aminoalkoxyalkyl and arylaminoalkyl; substituted amino such as alkylamino, alkylcarbonylanino, alkoxycarbonylamino, arylalkylamino, arylcarbonylamino, alkylthiocarbonylamino, arylsulfonylamino, heteroarylamino alkylcarbonylamino, arylcarbonylamino, heteroarylcarbonylamino, aralkylcarbonylamino, and heteroaralkylcarbonylamino; or substituted carbonyl such as unsubstituted or substituted aminocarbonyl, alkyloxycarbonyl, aryloxycarbonyl, aralkyloxycarbonyl and alkylaminoalkyloxycarbonyl. In other embodiments, R₆ may be selected from the group consisting of amidino, guanidino, cycloimido, heterocycloimido, cycloamido, heterocycloamido, cyclothioamido and heterocycloloweralkyl. In yet other embodiments, R₆ may be aryl or heteroaryl, such as, for example, substituted or unsubstituted pyridyl, pyrimidinyl, thiazolyl, indolyl, imidazolyl, oxadiazolyl, tetrazolyl, pyrazinyl, triazolyl, thienyl, furanyl, quinolinyl, pyrrolyopyridyl, benzothiazolyl, benzopyridyl, benzotriazolyl, and benzimidazolyl.

As used herein, representative heterocyclo groups include, for example, those shown below (where the point of attachment of the substituent group, and the other substituent groups shown below, is through the upper left-hand bond). These heterocyclo groups can be further substituted and may be attached at various positions as will be apparent to those having skill in the organic and medicinal chemistry arts in conjunction with the disclosure herein.

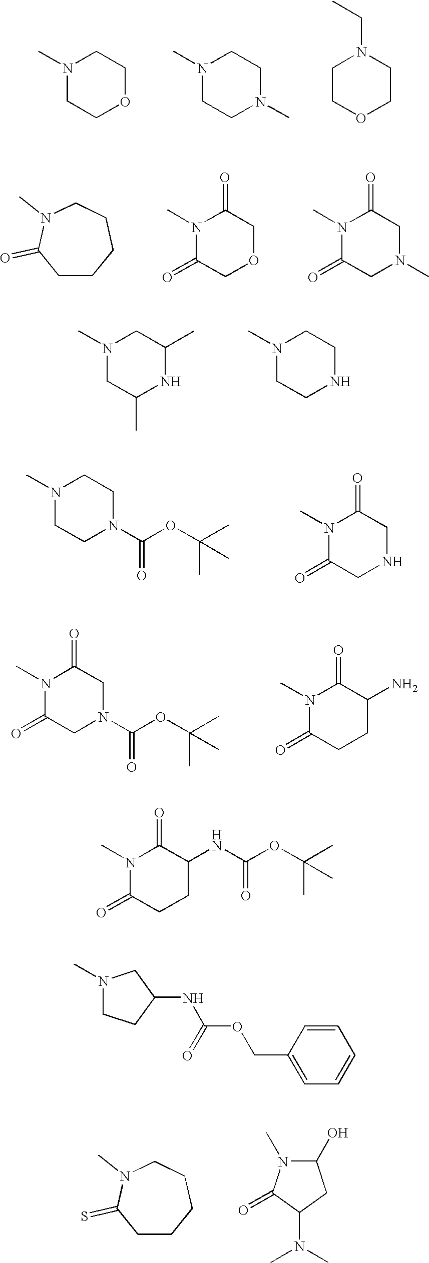

Representative heteroaryl groups include, for example, those shown below. These heteroaryl groups can be further substituted and may be attached at various positions as will be apparent to those having skill in the organic and medicinal chemistry arts in conjunction with the disclosure herein.

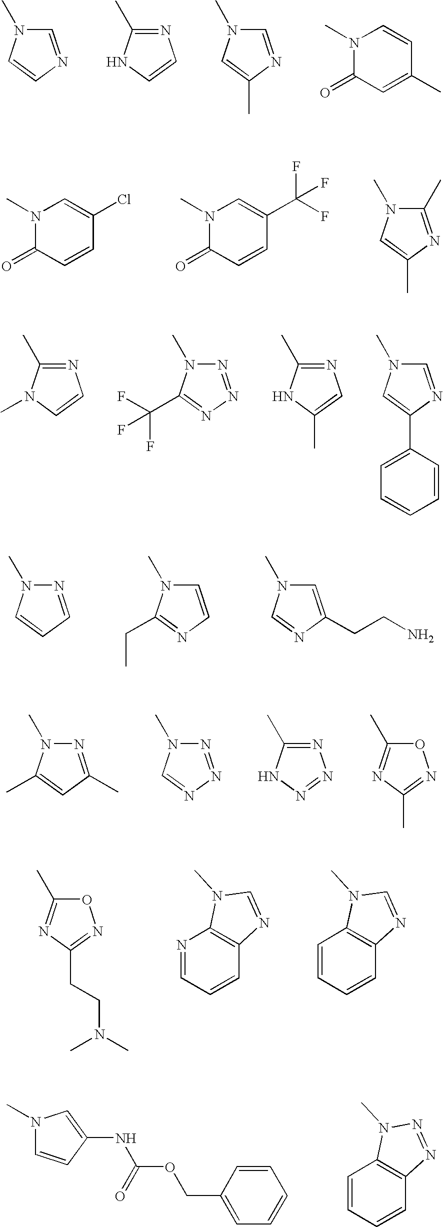

Representative cycloimido and heterocycloimido groups include, for example, those shown below. These cycloimido and heterocycloimido can be further substituted and may be attached at various positions as will be apparent to those having skill in the organic and medicinal chemistry arts in conjunction with the disclosure herein.

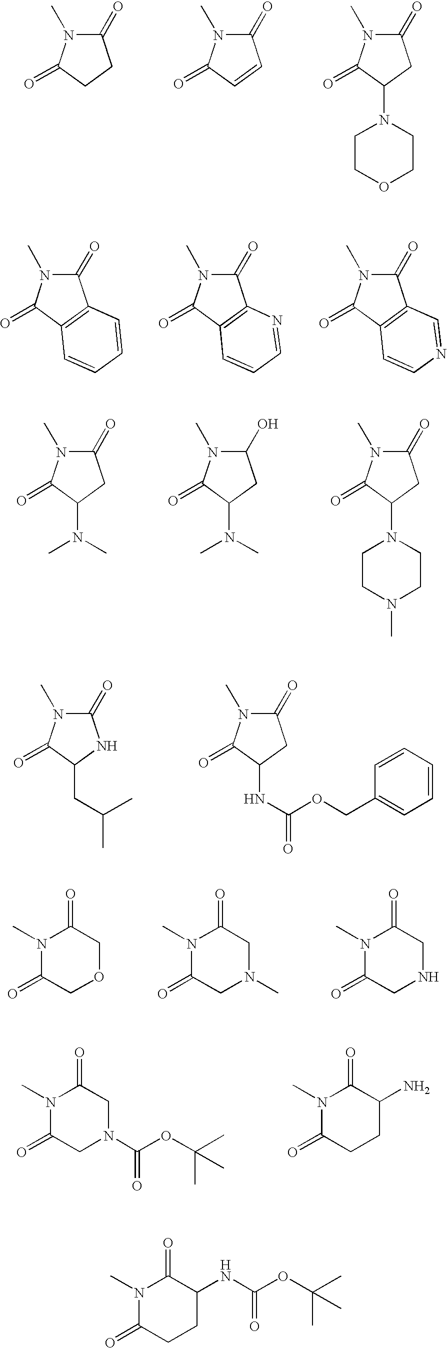

Representative substituted amidino and heterocycloamidino groups include, for example, those shown below. These amidino and heterocycloamidino groups can be further substituted as will be apparent to those having skill in the organic and medicinal chemistry arts in conjunction with the disclosure herein.

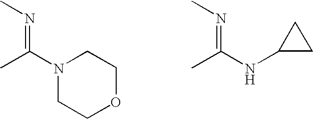

Representative substituted alkylcarbonylamino, alkyloxycarbonylamino, aminoalkyloxycarbonylamino, and arylcarbonylamino groups include, for example, those shown below. These groups can be further substituted as will be apparent to those having skill in the organic and medicinal chemistry arts in conjunction with the disclosure herein.

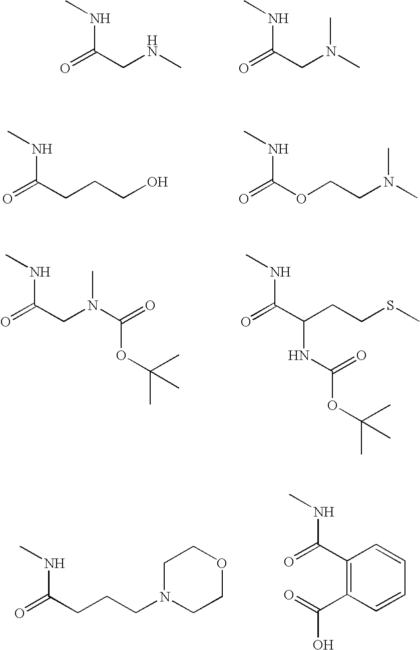

Representative substituted aminocarbonyl groups include, for example, those shown below. These can heterocyclo groups be further substituted as will be apparent to those having skill in the organic and medicinal chemistry arts in conjunction with the disclosure herein.

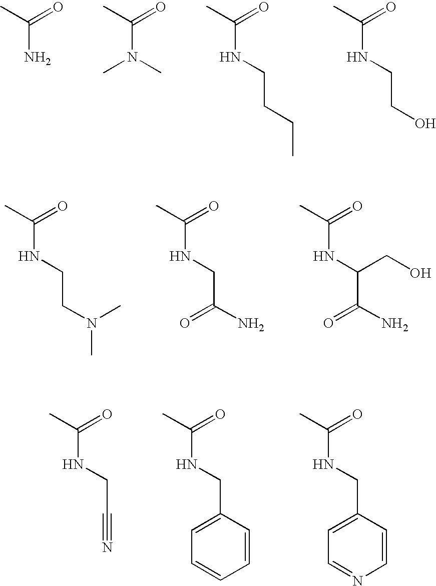

Representative substituted alkoxycarbonyl groups include, for example, those shown below. These alkoxycarbonyl groups can be further substituted as will be apparent to those having skill in the organic and medicinal chemistry arts in conjunction with the disclosure herein.

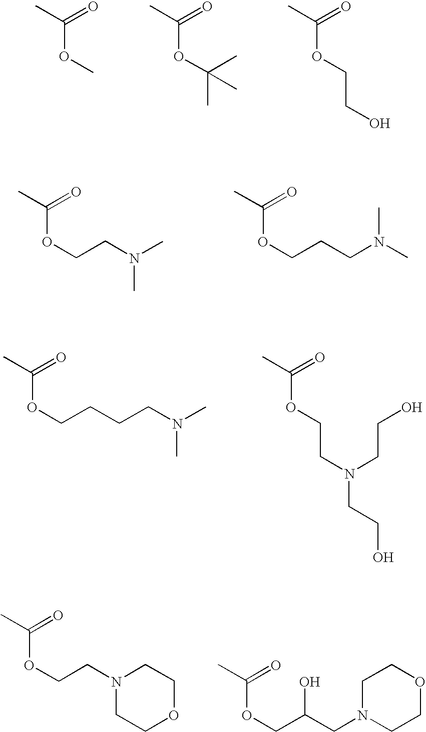

In some embodiments of the invention, GSK3 inhibitor compounds include compounds having the structure: 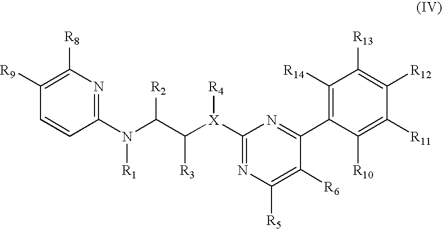
wherein X, R₁-R₆, and R₈-R₁₄ have the meanings described above, and the pharmaceutically acceptable salts thereof. Presently preferred, representative compounds of this group include, for example, [4-(4-imidazolylphenyl)pyrimidin-2-yl] {2-[(5-nitro(2-pyridyl))amino]ethyl}amine, 4-[5-imidazolyl-2-({2-[(5-nitro(2-pyridyl))amino]ethyl}-amino)pyrimidin-4-yl]benzenecarbonitrile, 4-[2-({2-[(6-amino-5-nitro(2-pyridyl))amino]-ethyl}amino)-5-imidazolylpyrimidin-4-yl]benzenecarbonitrile, [4-(2,4-dichlorophenyl)-5-imidazolylpyrimidin-2-yl]{2-[(5-nitro(2-pyridyl))amino]ethyl}amine, 4-[2-({2-[2-pyridyl)amino]ethyl}amino)-7a-hydro-1,2,4-triazolo[1,5-a]pyrimidin-7-yl]benzenecarbonitrile, {2-[(6-amino-5-nitro(2-pyridyl))amino]ethyl}[4-(2,4-dichlorophenyl)-5-imidazolylpyrimidin-2-yl]amine, [4-(2,4-dichlorophenyl)-5-imidazol-2-ylpyrimidin-2-yl]{2-[(5-nitro(2-pyridyl))amino]ethyl}amine, 6-[(2-{[4-(2,4-dichlorophenyl)-5-imidazolylpyrimidin-2-yl]amino}ethyl)amino]pyridine-3-carbonitrile, [5-benzotriazolyl-4-(2,4-dichlorophenyl)pyrimidin-2-yl]{2-[(5-nitro(2-pyridyl))amino]ethyl}amine, [2-({2-[(6-amino-5-nitro(2-pyridyl))amino]ethyl}amino)-4-(2,4-dichlorophenyl)pyrimidin-5-yl]-methan-1-ol, [4-(2,4-dichlorophenyl)-2-({2-[(5-nitro(2-pyridyl))amino]ethyl}amino)-pyrimidin-5-yl]methan-1-ol, 2-[2-({2-[(6-amino-5-nitro(2-pyridyl))amino]ethyl}amino)-4-(2,4-dichlorophenyl)pyrimidin-5-yl]isoindoline-1,3-dione, [5-amino-4-(2,4-dichlorophenyl)pyrimidin-2-yl]{2-[(6-amino-5-nitro(2-pyridyl))amino]ethyl}amine, {2-[(6-amino-5-nitro(2-pyridyl))amino]ethyl}[4-(2,4-dichlorophenyl)-5-morpholin-4-yl-pyrimidin-2-yl]amine, {2-[(6-amino-5-nitro(2-pyridyl))amino]ethyl}{4-(2,4-dichlorophenyl)-5-[5-(trifluoromethyl)(1,2,3,4-tetraazolyl)]pyrimidin-2-yl}amine, 1-[2-({2-[(6-amino-5-nitro(2-pyridyl))amino]ethyl}amino)-4-(2,4-dichlorophenyl)pyrimidin-5-yl]-pyrrolidine-2,5-dione, [4-(2,4-dichlorophenyl)-5-pyrazolylpyrimidin-2-yl]{2-[(5-nitro(2-pyridyl))amino]ethyl}amine, [4-(2,4-dichlorophenyl)-5-(4-methylimidazolyl)pyrimidin-2-yl]{2-[(5-nitro(2-pyridyl))amino]ethyl}amine, [4-(2,4-dichlorophenyl)-5-(2,4-dimethylimidazolyl)pyrimidin-2-yl]{2-[(5-nitro(2-pyridyl))amino]ethyl}amine, 6-[(2-{[4-(2,4-dichlorophenyl)-5-imidazol-2-ylpyrimidin-2-yl]amino}ethyl)amino]pyridine-3-carbonitrile, {2-[(6-amino-5-nitro(2-pyridyl))amino]ethyl}[4-(2,4-dichlorophenyl)-5-(morpholin-4-ylmethyl)pyrimidin-2-yl]amine, {2-[(6-amino-5-nitro(2-pyridyl))amino]-ethyl}[4-(2,4-dichlorophenyl)-5-piperazinylpyrimidin-2-yl]amine, {2-[(6-amino-5-nitro(2-pyridyl))amino]ethyl}[4-(4-ethylphenyl)-5-imidazolylpyrimidin-2-yl]amine, 1-[4-(2,4-dichlorophenyl)-2-({2-[(5-nitro(2-pyridyl))amino]ethyl}amino)pyrimidin-5-yl]hydropyridin-2-one, [5-benzimidazolyl-4-(2,4-dichlorophenyl)pyrimidin-2-yl]{2-[(5-nitro(2-pyridyl))amino]ethyl}amine, {2-[(6-amino-5-nitro(2-pyridyl))amino]ethyl}[4-(2,4-dichlorophenyl)-5-imidazolylpyrimidin-2-yl]methylamine, {2-[(6-amino-5-nitro(2-pyridyl))amino]ethyl}[4-(2,4-dichlorophenyl)-5-(4-pyridyl)pyrimidin-2-yl]amine, {2-[(6-amino-5-nitro(2-pyridyl))amino]ethyl}[4-(2,4-dichlorophenyl)-5-(4-methylpiperazinyl)-pyrimidin-2-yl]amine, [4-(2,4-dichlorophenyl)-5-(2-methylimidazolyl)pyrimidin-2-yl]{2-[(5-nitro(2-pyridyl))amino]ethyl}amine, {2-[(6-amino-5-nitro(2-pyridyl))amino]-ethyl}[4-(2,4-dichlorophenyl)-5-(2-methylimidazolyl)pyrimidin-2-yl]amine, {2-[(6-amino-5-nitro(2-pyridyl))amino]ethyl}[4-(2,4-dichlorophenyl)-5-(4-phenylimidazolyl)-pyrimidin-2-yl]amine, {2-[(6-amino-5-nitro(2-pyridyl))amino]ethyl}[4-(2,4-dichlorophenyl)-5-(2,4-dimethylimidazolyl)pyrimidin-2-yl]amine, [4-(2,4-dichlorophenyl)-5-imidazol-2-ylpyrimidin-2-yl](2-{[5-(trifluoromethyl)(2-pyridyl)]amino}ethyl)amine, [4-(2,4-dichlorophenyl)-5-piperazinylpyrimidin-2-yl]{2-[(5-nitro(2-pyridyl))amino]ethyl}-amine, [4-(2,4-dichlorophenyl)-5-imidazolylpyrimidin-2-yl] [2-(dimethylamino)ethyl]{2-[(5-nitro(2-pyridyl))amino]ethyl}amine, 1-[2-({2-[(6-amino-5-nitro(2-pyridyl))amino]-ethyl}amino)-4-(2,4-dichlorophenyl)pyrimidin-5-yl]-4-methylpiperazine-2,6-dione, [4-(2,4-dichlorophenyl)-5-(1-methylimidazol-2-yl)pyrimidin-2-yl]{2-[(5-nitro(2-pyridyl))amino]ethyl}amine, 1-[2-({2-[(6-amino-5-nitro(2-pyridyl))amino]ethyl}amino)-4-(2,4-dichlorophenyl)pyrimidin-5-yl]-3-morpholin-4-ylpyrrolidine-2,5-dione, 1-[4-(2,4-dichlorophenyl)-2-({2-[(5-nitro(2-pyridyl))amino]ethyl}amino)pyrimidin-5-yl]-4-methylpiperazine-2,6-dione, 1-[2-({2-[(6-amino-5-nitro(2-pyridyl))amino]ethyl}amino)-4-(2,4-dichlorophenyl)pyrimidin-5-yl]-3-(dimethylamino)pyrrolidine-2,5-dione, {5-imidazol-2-yl-4-[4-(trifluoromethyl)phenyl]pyrimidin-2-yl}{2-[(5-nitro(2-pyridyl))amino]-ethyl}amine, {2-[(6-amino-5-nitro(2-pyridyl))amino]ethyl}[4-(2,4-dichlorophenyl)-5-(1-methylimidazol-2-yl)pyrimidin-2-yl]amine, [4-(2,4-dichlorophenyl)-5-(4-methylpiperazinyl)pyrimidin-2-yl]{2-[(5-nitro(2-pyridyl))amino]ethyl}amine, [4-(2,4-dichlorophenyl)-5-(morpholin-4-ylmethyl)pyrimidin-2-yl]{2-[(5-nitro(2-pyridyl))amino]ethyl}-amine, [4-(2,4-dichlorophenyl)-5-(4-methylimidazol-2-yl)pyrimidin-2-yl]{2-[(5-nitro(2-pyridyl))amino]ethyl}amine, {2-[(6-amino-5-nitro(2-pyridyl))amino]ethyl}[4-(2,4-dichlorophenyl)-5-(4-methylimidazol-2-yl)pyrimidin-2-yl]amine, (2-[(6-amino-5-nitro(2-pyridyl))amino]ethyl}[4-(2-chlorophenyl)-5-imidazol-2-ylpyrimidin-2-yl]amine, [4-(2-chloro-4-fluorophenyl)-5-imidazol-2-ylpyrimidin-2-yl]{2-[(5-nitro(2-pyridyl))amino]ethyl}amine, [4-(2,4-dichlorophenyl)-5-imidazolylpyrimidin-2-yl]{2-[(5-nitro(2-pyridyl))amino]ethyl}(2-pyrrolidinylethyl)amine, [4-(2,4-dichlorophenyl)-5-imidazolylpyrimidin-2-yl](2-morpholin-4-ylethyl){2-[(5-nitro(2-pyridyl))amino]ethyl}amine, 6-[(2-{[4-(2,4-dichlorophenyl)-5-(4-methylimidazol-2-yl)pyrimidin-2-yl]amino}ethyl)amino]-pyridine-3-carbonitrile, {2-[(6-amino-5-nitro(2-pyridyl))amino]ethyl}[4-(2-chloro-4-fluorophenyl)-5-imidazol-2-ylpyrimidin-2-yl]amine, [4-(4-ethylphenyl)-5-imidazol-2-ylpyrimidin-2-yl]{2-[(5-nitro(2-pyridyl))amino]ethyl}amine, [5-((1E)-1-aza-2-morpholin-4-ylprop-1-enyl)-4-(2,4-dichlorophenyl)pyrimidin-2-yl]{2-[(6-amino-5-nitro(2-pyridyl))amino]ethyl}amine, N-[4-(2,4-dichlorophenyl)-2-({2-[(5-nitro(2-pyridyl))amino]ethyl}amino)pyrimidin-5-yl]acetamide, [4-(2,4-dichlorophenyl)-5-imidazol-2-ylpyrimidin-2-yl]{2-[(6-methoxy-5-nitro(2-pyridyl))amino]ethyl}amine, 6-[(2-{[4-(2,4-dichlorophenyl)-5-imidazolylpyrimidin-2-yl]methylamino}ethyl)amino]-pyridine-3-carbonitrile, 6-[(2-{[4-(2,4-dichlorophenyl)-5-imidazol-2-ylpyrimidin-2-yl]methylamino}ethyl)amino]pyridine-3-carbonitrile, [4-(2,4-dichlorophenyl)-5-imidazol-2-ylpyrimidin-2-yl]methyl{2-[(5-nitro(2-pyridyl))amino]ethyl}amine, 6-[(2-{[4-(2-chloro-4-fluorophenyl)-5-imidazol-2-ylpyrimidin-2-yl]amino}ethyl)amino]-pyridine-3-carbonitrile, [4-(4-chlorophenyl)-5-imidazol-2-ylpyrimidin-2-yl]{2-[(5-nitro-(2-pyridyl))amino]ethyl}amine, {2-[(6-amino-5-nitro(2-pyridyl))amino]ethyl}[4-(4-chloro-2-methylphenyl)-5-imidazol-2-ylpyrimidin-2-yl]amine, {2-[(6-amino-5-nitro(2-pyridyl))amino]ethyl}[4-(4-bromo-2-chlorophenyl)-5-imidazol-2-ylpyrimidin-2-yl]-amine, 6-[(2-{[4-(4-bromo-2-chlorophenyl)-5-imidazol-2-ylpyrimidin-2-yl]amino}ethyl)-amino]pyridine-3-carbonitrile, 6-[2-({2-[(6-amino-5-nitro(2-pyridyl))amino]ethyl}-amino)-4-(2,4-dichlorophenyl)pyrimidin-5-yl]-3-pyrrolino[3,4-b]pyridine-5,7-dione, N-[2-({2-[(6-amino-5-nitro(2-pyridyl))amino]ethyl}amino)-4-(2,4-dichlorophenyl)-pyrimidin-5-yl]-2-(methylamino)acetamide, {2-[(6-amino-5-nitro(2-pyridyl))amino]-ethyl}[4-(4-bromo-2-chlorophenyl)-5-(4-methylimidazol-2-yl)pyrimidin-2-yl]amine, 6-[(2-{[4-(4-bromo-2-chlorophenyl)-5-(4-methylimidazol-2-yl)pyrimidin-2-yl]amino}-ethyl)amino]pyridine-3-carbonitrile, {2-[(6-amino-5-nitro(2-pyridyl))amino]ethyl}[4-(2-chloro-4-fluorophenyl)-5-(4-methylimidazol-2-yl)pyrimidin-2-yl]amine, and 6-[(2-{[4-(2,4-dichlorophenyl)-5-(5-chloro-2-oxohydropyridyl)pyrimidin-2-yl]amino}ethyl)-amino]pyridine-3-carbonitrile.

Other presently particularly preferred compounds of the invention include compounds having the structure: 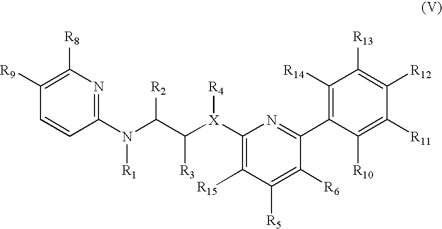
wherein X, R₁-R₆, and R₈-R₁₄ have the meanings described above, and R₁₅ is selected from the group consisting of hydrogen, nitro, cyano, amino, alkyl, halo, haloloweralkyl, alkyloxycarbonyl, aminocarbonyl, alkylsulfonyl and arylsulfonyl, and the pharmaceutically acceptable salts thereof. Presently preferred, representative compounds of this group include, for example, [6-(2,4-dichlorophenyl)-5-imidazolyl(2-pyridyl)]{2-[(5-nitro(2-pyridyl))amino]ethyl}amine, {2-[(6-amino-5-nitro(2-pyridyl))amino]ethyl}[6-(2,4-dichlorophenyl)-5-imidazolyl(2-pyridyl)]amine, 6-[(2-{[6-(2,4-dichlorophenyl)-5-imidazolyl-2-pyridyl]amino}ethyl)amino]pyridine-3-carbonitrile, {2-[(6-amino-5-nitro(2-pyridyl))amino]ethyl}[6-(2,4-dichlorophenyl)-5-nitro(2-pyridyl)]amine, {2-[(6-amino-5-nitro(2-pyridyl))amino]ethyl}[6-(2,4-dichlorophenyl)-5-(4-methylimidazolyl)(2-pyridyl)]amine, 6-[(2-{[6-(2,4-dichlorophenyl)-5-(4-methylimidazolyl)-2-pyridyl]-amino}ethyl)amino]pyridine-3-carbonitrile, and [4-(4-bromo-2-chlorophenyl)-5-imidazol-2-ylpyrimidin-2-yl]{2-[(5-nitro(2-pyridyl))amino]ethyl}amine.

In other embodiments, the GSK3 inhibitor compounds used in the practice of the invention include the GSK3 inhibitor compounds as disclosed in U.S. and international patent application Publication Nos. 20020156087, WO0220495 and WO9965897 (pyrimidine and pyridine based compounds); 20030008866, 20010044436 and WO0144246 (bicyclic based compounds); 20010034051 (pyrazine based compounds); and WO9816528 (purine based compounds). Furthermore, additional GSK3 inhibitory compounds useful within the context of this invention include those disclosed in WO0222598 (quinolinone based compounds). The entire disclosure of these international publications is incorporated herein by this reference.

In another aspect, the present invention provides therapeutic compositions comprising at least one GSK3 inhibitor compound in combination with one or more additional agents for the treatment of ischemic stroke, as are commonly employed in stroke therapy. Useful additional agents include, for example, thrombolytic and/or fibrinolytic agents that help reestablish cerebral circulation by dissolving (lysing) the clots which obstruct blood flow, neuroprotective agents that work to minimize the effects of the ischemic cascade, anticoagulants and antiplatelet agents. Representative thrombolytic agents include, for example, alteplase (tissue plasminogen activator (t-PA)), an enzyme found naturally in the body which converts, or activates, plasminogen into the enzyme plasmin to dissolve a blood clot; anistreplase; reteplase; urokinase; and streptokinase. Representative neuroprotective agents include, for example, glutamate antagonists that interfere with the progression of glutamate into the neurons, calcium antagonists that block the intracellular build-up of calcium through electrically-operated channels, opiate antagonists that interfere with the ischemic cascade by working on the opiate receptors which are overstimulated by the chemical cascade that occurs during cellular death, GABA-A agonists such as Clomethiazole (Astra) that activate the GABA-A receptor and thereby counteract the electrical activity of certain glutamate receptors, calpain inhibitors, NMDA receptor antagonists such as C-101,606 (Pfizer), K⁺ channel modulators such as BMS-204352 (Bristol-Myers Squibb), PDH kinase inhibitors, and antioxidants that scavenge free radicals generated by the ischemic cascade. In addition, the methods and compounds of the invention may be used in combination with oxygenated fluorocarbon nutrient emulsion (OFNE) therapy and/or neuroperfusion in which oxygen-rich blood is rerouted through the brain to reduce damage from an ischemic stroke. Representative anticoagulants include, for example, heparin, warfarin, dalteparin, danaparoid, enoxaparin, tinzaparin, 4-hydroxycoumarin, dicumarol, phenprocoumon, acenocoumarol, anisindone, lepirudin -and indane-1,3-dione. Representative antiplatelet agents include, for example, aspirin, clopidogrel, ticlopidine, abciximab, eptifibatide, tirofiban and dipyridamole.

As used above and elsewhere herein the following terms have the meanings defined below:

“Glycogen synthase kinase 3” and “GSK3” are used interchangeably herein to refer to any protein having more than 60% sequence homology to the amino acids between positions 56 and 340 of the human GSK3 beta amino acid sequence (Genbank Accession No. L33801). To determine the percent homology of two amino acid sequences or of two nucleic acids, the sequences are aligned for optimal comparison purposes (e.g., gaps can be introduced in the sequence of one polypeptide or nucleic acid for optimal alignment with the other polypeptide or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100). GSK3 was originally identified by its phosphorylation of glycogen synthase as described in Woodgett et al., Trends Biochem. Sci., 16:177-81 (1991), incorporated herein by reference. By inhibiting GSK3 kinase activity, activities downstream of GSK3 activity may be inhibited, or, alternatively, stimulated. For example, when GSK3 activity is inhibited, glycogen synthase may be activated, resulting in increased glycogen production. GSK3 is also known to act as a kinase in a variety of other contexts, including, for example, phosphorylation of c-jun, β-catenin, and tau protein. It is understood that inhibition of GSK3 kinase activity can lead to a variety of effects in a variety of biological contexts. The invention, however, is not limited by any theories of mechanism as to how the invention works.

“GSK3 inhibitor” is used herein to refer to a compound that exhibits an IC₅₀ with respect to GSK3 of no more than about 100 μM and more typically not more than about 50 μM, as measured in the cell-free assay for GSK3 inhibitory activity described generally hereinbelow. “IC₅₀” is that concentration of inhibitor which reduces the activity of an enzyme (e.g., GSK3) to half-maximal level. Representative compounds of the present invention have been discovered to exhibit inhibitory activity against GSK3. Compounds of the present invention preferably exhibit an IC₅₀ with respect to GSK3 of no more than about 10 μM, more preferably, no more than about 5 μM, even more preferably not more than about 1 μM, and most preferably, not more than about 200 nM, as measured in the cell-free GSK3 kinase assay.

“Optionally substituted” refers to the replacement of hydrogen with a monovalent or divalent radical. Suitable substitution groups include, for example, hydroxyl, nitro, amino, imino, cyano, halo, thio, thioamido, amidino, imidino, oxo, oxamidino, methoxamidino, imidino, guanidino, sulfonamido, carboxyl, formyl, loweralkyl, haloloweralkyl, loweralkoxy, haloloweralkoxy, loweralkoxyalkyl, alkylcarbonyl, arylcarbonyl, aralkylcarbonyl, heteroarylcarbonyl, heteroaralkylcarbonyl, alkylthio, aminoalkyl, cyanoalkyl, and the like.

The substitution group can itself be substituted. The group substituted onto the substitution group can be carboxyl, halo; nitro, amino, cyano, hydroxyl, loweralkyl, loweralkoxy, aminocarbonyl, —SR, thioamido, —SO₃H, —SO₂R or cycloalkyl, where R is typically hydrogen, hydroxyl or loweralkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substitutents can be straight chain, branched or cyclic arrangements of covalently bonded carbon or heteroatoms.

“Loweralkyl” as used herein refers to branched or straight chain alkyl groups comprising one to ten carbon atoms that are unsubstituted or substituted, e.g., with one or more halogen, hydroxyl or other groups, including, e.g., methyl, ethyl, propyl, isopropyl, n-butyl, t-butyl, neopentyl, trifluoromethyl, pentafluoroethyl and the like.

“Alkylenyl” refers to a divalent straight chain or branched chain saturated aliphatic radical having from 1 to 20 carbon atoms. Typical alkylenyl groups employed in compounds of the present invention are loweralkylenyl groups that have from 1 to about 6 carbon atoms in their backbone. “Alkenyl” refers herein to straight chain, branched, or cyclic radicals having one or more double bonds and from 2 to 20 carbon atoms. “Alkynyl” refers herein to straight chain, branched, or cyclic radicals having one or more triple bonds and from 2 to 20 carbon atoms.

“Loweralkoxy” as used herein refers to RO— wherein R is loweralkyl. Representative examples of loweralkoxy groups include methoxy, ethoxy, t-butoxy, trifluoromethoxy and the like.

“Cycloalkyl” refers to a mono- or polycyclic, heterocyclic or carbocyclic alkyl substituent. Typical cycloalkyl substituents have from 3 to 8 backbone (i.e., ring) atoms in which each backbone atom is either carbon or a heteroatom. The term “heterocycloalkyl” refers herein to cycloalkyl substituents that have from 1 to 5, and more typically from 1 to 4 heteroatoms in the ring structure. Suitable heteroatoms employed in compounds of the present invention are nitrogen, oxygen, and sulfur. Representative heterocycloalkyl moieties include, for example, morpholino, piperazinyl, piperadinyl and the like. Carbocycloalkyl groups are cycloalkyl groups in which all ring atoms are carbon. When used in connection with cycloalkyl substituents, the term “polycyclic” refers herein to fused and non-fused alkyl cyclic structures.

“Halo” refers herein to a halogen radical, such as fluorine, chlorine, bromine or iodine. “Haloalkyl” refers to an alkyl radical substituted with one or more halogen atoms. The term “haloloweralkyl” refers to a loweralkyl radical substituted with one or more halogen atoms. The term “haloalkoxy” refers to an alkoxy radical substituted with one or more halogen atoms. The term “haloloweralkoxy” refers to a loweralkoxy radical substituted with one or more halogen atoms.

“Aryl” refers to monocyclic and polycyclic aromatic groups having from 3 to 14 backbone carbon or hetero atoms, and includes both carbocyclic aryl groups and heterocyclic aryl groups. Carbocyclic aryl groups are aryl groups in which all ring atoms in the aromatic ring are carbon. The term “heteroaryl” refers herein to aryl groups having from 1 to 4 heteroatoms as ring atoms in an aromatic ring with the remainder of the ring atoms being carbon atoms. When used in connection with aryl substituents, the term “polycyclic” refers herein to fused and non-fused cyclic structures in which at least one cyclic structure is aromatic, such as, for example, benzodioxozolo (which has a heterocyclic structure fused to a phenyl group, i.e. 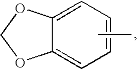
naphthyl, and the like. Exemplary aryl moieties employed as substituents in compounds of the present invention include phenyl, pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, and the like.

“Aralkyl” refers to an alkyl group substituted with an aryl group. Typically, aralkyl groups employed in compounds of the present invention have from 1 to 6 carbon atoms incorporated within the alkyl portion of the aralkyl group. Suitable aralkyl groups employed in compounds of the present invention include, for example, benzyl, picolyl, and the like.

“Amino” refers herein to the group —NH₂. The term “alkylamino” refers herein to the group —NRR′ where R and R′ are each independently selected from hydrogen or a lower alkyl. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl.

The term “arylcycloalkylamino” refers herein to the group, aryl-cycloalkyl-NH—, where cycloalkyl is a divalent cycloalkyl group. Typically, cycloalkyl has from 3 to 6 backbone atoms, of which, optionally 1 to about 4 are heteroatoms. The term “aminoalkyl” refers to an alkyl group that is terminally substituted with an amino group.

The term “alkoxyalkyl” refers to the group -alk₁-O-alk₂ where alk₁ is alkylenyl or alkenyl, and alk₂ is alkyl or alkenyl. The term “loweralkoxyalkyl” refers to an alkoxyalkyl where alk₁ is loweralkylenyl or loweralkenyl, and alk2 is loweralkyl or loweralkenyl. The term “aryloxyalkyl” refers to the group -alkylenyl-O-aryl. The term “aralkoxyalkyl” refers to the group -alkylenyl-O-aralkyl, where aralkyl is a loweraralkyl.

The term “alkoxyalkylamino” refers herein to the group —NR-(alkoxylalkyl), where R is typically hydrogen, loweraralkyl, or loweralkyl. The term “aminoloweralkoxyalkyl” refers herein to an aminoalkoxyalkyl in which the alkoxyalkyl is a loweralkoxyalkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. The term “arylaminocarbonyl” refers herein to the group —C(O)—NRR′ where R is an aryl and R′ is hydrogen, loweralkyl or aryl. “aralkylaminocarbonyl” refers herein to the group —C(O)—NRR′ where R is loweraralkyl and R′ is hydrogen, loweralkyl, aryl, or loweraralkyl.

“Aminosulfonyl” refers herein to the group —S(O)₂—NH₂. “Substituted aminosulfonyl” refers herein to the group —S(O)₂—NRR′ where R is loweralkyl and R′ is hydrogen or a loweralkyl. The term “aralkylaminosulfonlyaryl” refers herein to the group -aryl-S(O)₂—NH-aralkyl, where the aralkyl is loweraralkyl.

“Carbonyl” refers to the divalent group —C(O)—.

“Carbonyloxy” refers generally to the group —C(O)—O—. Such groups include esters, —C(O)—O—R, where R is loweralkyl, cycloalkyl, aryl, or loweraralkyl. The term “carbonyloxycycloalkyl” refers generally herein to both an “carbonyloxycarbocycloalkyl” and an “carbonyloxyheterocycloalkyl”, i.e., where R is a carbocycloalkyl or heterocycloalkyl, respectively. The term “arylcarbonyloxy” refers herein to the group —C(O)—O-aryl, where aryl is a mono- or polycyclic, carbocycloaryl or heterocycloaryl. The term “aralkylcarbonyloxy” refers herein to the group —C(O)—O-aralkyl, where the aralkyl is loweraralkyl.

The term “sulfonyl” refers herein to the group —SO₂—. “Alkylsulfonyl” refers to a substituted sulfonyl of the structure —SO₂R— in which R is alkyl. Alkylsulfonyl groups employed in compounds of the present invention are typically loweralkylsulfonyl groups having from 1 to 6 carbon atoms in its backbone structure. Thus, typical alkylsulfonyl groups employed in compounds of the present invention include, for example, methylsulfonyl (i.e., where R is methyl), ethylsulfonyl (i.e., where R is ethyl), propylsulfonyl (i.e., where R is propyl), and the like. The term “arylsulfonyl” refers herein to the group —SO₂-aryl. The term “aralkylsulfonyl” refers herein to the group —SO₂-aralkyl, in which the aralkyl is loweraralkyl. The term “sulfonamido” refers herein to —SO₂NH₂.

As used herein, the term “carbonylamino” refers to the divalent group —NH—C(O)— in which the hydrogen atom of the amide nitrogen of the carbonylamino group can be replaced a loweralkyl, aryl, or loweraralkyl group. Such groups include moieties such as carbamate esters (—NH—C(O)—O—R) and amides —NH—C(O)—O—R, where R is a straight or branched chain loweralkyl, cycloalkyl, or aryl or loweraralkyl. The term “loweralkylcarbonylamino” refers to alkylcarbonylamino where R is a loweralkyl having from 1 to about 6 carbon atoms in its backbone structure. The term “arylcarbonylamino” refers to group —NH—C(O)—R where R is an aryl. Similarly, the term “aralkylcarbonylamino ” refers to carbonylamino where R is a lower aralkyl.

As used herein, the term “guanidino” or “guanidyl” refers to moieties derived from guanidine, H₂N—C(═NH)—NH₂. Such moieties include those bonded at the nitrogen atom carrying the formal double bond (the “2”-position of the guanidine, e.g., diaminomethyleneamino, (H₂N)₂C═NH—) and those bonded at either of the nitrogen atoms carrying a formal single bond (the “1-” and/or “3”-positions of the guandine, e.g., H₂N—C(═NH)—NH—). The hydrogen atoms at any of the nitrogens can be replaced with a suitable substituent, such as loweralkyl, aryl, or loweraralkyl.

As used herein, the term “amidino” refers to the moieties R—C(═N)—NR′— (the radical being at the “N¹” nitrogen) and R(NR′)C═N— (the radical being at the “N²” nitrogen), where R and R′ can be hydrogen, loweralkyl, aryl, or loweraralkyl.

Compounds of the present invention can be readily synthesized using the methods described herein, or other methods, which are well known in the art. For example, the synthesis of pyrimidine and pyrimidine based compounds of the formula (I) above can be readily synthesized as described in international patent application Publication No. WO99-65897, published Dec. 23, 1999, incorporated herein by reference.

Compounds of the present invention preferably exhibit inhibitory activity that is relatively substantially selective with respect to GSK3, as compared to at least one other type of kinase. As used herein, the term “selective” refers to a relatively greater potency for inhibition against GSK3, as compared to at least one other type of kinase. Preferably, GSK3 inhibitors of the present invention are selective with respect to GSK3, as compared to at least two other types of kinases. Kinase activity assays for kinases other than GSK3 are generally known. See e.g., Havlicek et al., J. Med. Chem., 40:408-12 (1997), incorporated herein by reference. GSK3 selectivity can be quantitated according to the following: GSK3 selectivity=IC_{50 (other kinase)}÷IC_{50 (GSK3)}, where a GSK3 inhibitor is selective for GSK3 when IC_{50 (other kinase)}>IC_{50 (GSK3)}. Thus, an inhibitor that is selective for GSK3 exhibits a GSK3 selectivity of greater than 1-fold with respect to inhibition of a kinase other than GSK3. As used herein, the term “other kinase” refers to a kinase other than GSK3. Such selectivities are generally measured in the cell-free assay below.

Typically, GSK3 inhibitors of the present invention exhibit a selectivity of at least about 2-fold (i.e., IC_{50 (other kinase)}÷IC_{50 (GSK3)}) for GSK3, as compared to another kinase and more typically they exhibit a selectivity of at least about 5-fold. Usually, GSK3 inhibitors of the present invention exhibit a selectivity for GSK3, as compared to at least one other kinase, of at least about 10-fold, desirably at least about 100-fold, and more preferably, at least about 1000-fold.

GSK3 inhibitory activity can be readily detected using the assays described herein, as well as assays generally known to those of ordinary skill in the art. Exemplary methods for identifying specific inhibitors of GSK3 include both cell-free and cell-based GSK3 kinase assays. A cell-free GSK3 kinase assay detects inhibitors that act by direct interaction with the polypeptide GSK3, while a cell-based GSK3 kinase assay may identify inhibitors that function either by direct interaction with GSK3 itself, or by interference with GSK3 expression or with post-translational processing required to produce mature active GSK3.

In general, a cell-free GSK3 kinase assay can be readily carried out by: (1) incubating GSK3 with a peptide substrate, radiolabeled ATP (such as, for example, γ³³P- or γ³²P-ATP, both available from Amersham, Arlington Heights, Ill.), magnesium ions, and optionally, one or more candidate inhibitors; (2) incubating the mixture for a period of time to allow incorporation of radiolabeled phosphate into the peptide substrate by GSK3 activity; (3) transferring all or a portion of the enzyme reaction mix to a separate vessel, typically a microtiter well that contains a uniform amount of a capture ligand that is capable of binding to an anchor ligand on the peptide substrate; (4) washing to remove unreacted radiolabeled ATP; then (5) quantifying the amount of ³³P or ³²P remaining in each well. This amount represents the amount of radiolabeled phosphate incorporated into the peptide substrate. Inhibition is observed as a reduction in the incorporation of radiolabeled phosphate into the peptide substrate.

Suitable peptide substrates for use in the cell free assay may be any peptide, polypeptide or synthetic peptide derivative that can be phosphorylated by GSK3 in the presence of an appropriate amount of ATP. Suitable peptide substrates may be based on portions of the sequences of various natural protein substrates of GSK3, and may also contain N-terminal or C-terminal modifications or extensions including spacer sequences and anchor ligands. Thus, the peptide substrate may reside within a larger polypeptide, or may be an isolated peptide designed for phosphorylation by GSK3.

For example, a peptide substrate can be designed based on a subsequence of the DNA binding protein CREB, such as the SGSG-linked CREB peptide sequence within the CREB DNA binding protein described in Wang et al., Anal. Biochem., 220:397-402 (1994), incorporated herein by reference. In the assay reported by Wang et al., the C-terminal serine in the SXXXS motif of the CREB peptide is enzymatically prephosphorylated by cAMP-dependent protein kinase (PKA), a step which is required to render the N-terminal serine in the motif phosphorylatable by GSK3. As an alternative, a modified CREB peptide substrate can be employed which has the same SXXXS motif and which also contains an N-terminal anchor ligand, but which is synthesized with its C-terminal serine prephosphorylated (such a substrate is available commercially from Chiron Technologies PTY Ltd., Clayton, Australia). Phosphorylation of the second serine in the SXXXS motif during peptide synthesis eliminates the need to enzymatically phosphorylate that residue with PKA as a separate step, and incorporation of an anchor ligand facilitates capture of the peptide substrate after its reaction with GSK3.

Generally, a peptide substrate used for a kinase activity assay may contain one or more sites that are phosphorylatable by GSK3, and one or more other sites that are phosphorylatable by other kinases, but not by GSK3. Thus, these other sites can be prephosphorylated in order to create a motif that is phosphorylatable by GSK3. The term “prephosphorylated” refers herein to the phosphorylation of a substrate peptide with non-radiolabeled phosphate prior to conducting a kinase assay using that substrate peptide. Such prephosphorylation can conveniently be performed during synthesis of the peptide substrate.

The SGSG-linked CREB peptide can be linked to an anchor ligand, such as biotin, where the serine near the C terminus between P and Y is prephosphorylated. As used herein, the term “anchor ligand” refers to a ligand that can be attached to a peptide substrate to facilitate capture of the peptide substrate on a capture ligand, and which functions to hold the peptide substrate in place during wash steps, yet allows removal of unreacted radiolabeled ATP. An exemplary anchor ligand is biotin. The term “capture ligand” refers herein to a molecule which can bind an anchor ligand with high affinity, and which is attached to a solid structure. Examples of bound capture ligands include, for example, avidin- or streptavidin-coated microtiter wells or agarose beads. Beads bearing capture ligands can be further combined with a scintillant to provide a means for detecting captured radiolabeled substrate peptide, or scintillant can be added to the captured peptide in a later step.

The captured radiolabeled peptide substrate can be quantitated in a scintillation counter using known methods. The signal detected in the scintillation counter will be proportional to GSK3 activity if the enzyme reaction has been run under conditions where only a limited portion (e.g., less than 20%) of the peptide substrate is phosphorylated. If an inhibitor is present during the reaction, GSK3 activity will be reduced, and a smaller quantity of radiolabeled phosphate will thus be incorporated into the peptide substrate. Hence, a lower scintillation signal will be detected. Consequently, GSK3 inhibitory activity will be detected as a reduction in scintillation signal, as compared to that observed in a negative control where no inhibitor is present during the reaction.

A cell-based GSK3 kinase activity assay typically utilizes a cell that can express both GSK3 and a GSK3 substrate, such as, for example, a cell transformed with genes encoding GSK3 and its substrate, including regulatory control sequences for the expression of the genes. In carrying out the cell-based assay, the cell capable of expressing the genes is incubated in the presence of a compound of the present invention. The cell is lysed, and the proportion of the substrate in the phosphorylated form is determined, e.g., by observing its mobility relative to the unphosphorylated form on SDS PAGE or by determining the amount of substrate that is recognized by an antibody specific for the phosphorylated form of the substrate. The amount of phosphorylation of the substrate is an indication of the inhibitory activity of the compound, i.e., inhibition is detected as a decrease in phosphorylation as compared to the assay conducted with no inhibitor present. GSK3 inhibitory activity detected in a cell-based assay may be due, for example, to inhibition of the expression of GSK3 or by inhibition of the kinase activity of GSK3.

Thus, cell-based assays can also be used to specifically assay for activities that are implicated by GSK3 inhibition, such as, for example, inhibition of tau protein phosphorylation, potentiation of insulin signaling, and the like. For example, to assess the capacity of a GSK3 inhibitor to inhibit Alzheimer's-like phosphorylation of microtubule-associated protein tau, cells may be co-transfected with human GSK3β and human tau protein, then incubated with one or more candidate inhibitors. Various mammalian cell lines and expression vectors can be used for this type of assay. For instance, COS cells may be transfected with both a human GSK3β expression plasmid according to the protocol described in Stambolic et al., 1996, Current Biology 6:1664-68, which is incorporated herein by reference, and an expression plasmid such as pSG5 that contains human tau protein coding sequence under an early SV40 promoter. See also Goedert et al., EMBO J., 8:393-399 (1989), which is incorporated herein by reference. Alzheimer's-like phosphorylation of tau can be readily detected with a specific antibody such as, for example, AT8, which is available from Polymedco Inc. (Cortlandt Manor, N.Y.) after lysing the cells. This assay is described in greater detail in the examples, hereinbelow.

Likewise, the ability of GSK3 inhibitor compounds to potentiate insulin signaling by activating glycogen synthase can be readily ascertained using a cell-based glycogen synthase activity assay. This assay employs cells that respond to insulin stimulation by increasing glycogen synthase activity, such as the CHO-HIRC cell line, which overexpresses wild-type insulin receptor (˜100,000 binding sites/cell). The CHO-HIRC cell line can be generated as described in Moller et al., J. Biol. Chem., 265:14979-14985 (1990) and Moller et al., Mol. Endocrinol., 4:1183-1191 (1990), both of which are incorporated herein by reference. The assay can be carried out by incubating serum-starved CHO-HIRC cells in the presence of various concentrations of compounds of the present invention in the medium, followed by cell lysis at the end of the incubation period. Glycogen synthase activity can be detected in the lysate as described in Thomas et al., Anal. Biochem., 25:486-499 (1968). Glycogen synthase activity is computed for each sample as a percentage of maximal glycogen synthase activity, as described in Thomas et al., supra, and is plotted as a function of candidate GSK3 inhibitor concentration. The concentration of candidate GSK3 inhibitor that increased glycogen synthase activity to half of its maximal level (i.e., the EC₅₀) can be calculated by fitting a four parameter sigmoidal curve using routine curve fitting methods that are well known to those having ordinary skill in the art. This is described in more detail in Example 1, hereinbelow.

GSK3 inhibitors can be readily screened for in vivo activity such as, for example, using methods that are well known to those having ordinary skill in the art. For example, candidate compounds having potential therapeutic activity in the treatment of type 2 diabetes can be readily identified by detecting a capacity to improve glucose tolerance in animal models of type 2 diabetes. Specifically, the candidate compound can be dosed using any of several routes prior to administration of a glucose bolus in either diabetic mice (e.g. KK, db/db, ob/ob) or diabetic rats (e.g. Zucker Fa/Fa or GK). Following administration of the candidate compound and glucose, blood samples are removed at preselected time intervals and evaluated for serum glucose and insulin levels. Improved disposal of glucose in the absence of elevated secretion levels of endogenous insulin can be considered as insulin sensitization and can be indicative of compound efficacy. A detailed description of this assay is provided in the examples, hereinbelow.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, sulfate, 3-phenylpropionate, picrate, pivalate, propionate, succinate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

Compounds of the present invention can be administered in a variety of ways including enteral, parenteral, inhalation and topical routes of administration. For example, suitable modes of administration include oral, subcutaneous, transdermal, transmucosal, iontophoretic, intracerebral, intravenous, intraarterial, intramuscular, intraperitoneal, intranasal, intrathecal, subdural, rectal, and the like.

In accordance with other embodiments of the present invention, there is provided a composition comprising GSK3-inhibitor compound of the present invention, together with a pharmaceutically acceptable carrier or excipient.

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

Pharmaceutical compositions containing GSK-3 inhibitor compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

The compounds of the present invention may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In accordance with yet other embodiments, the present invention provides methods for inhibiting ischemic damage or injury due to GSK3 activity in a human or animal subject, said method comprising administering to a subject an amount of a GSK3 inhibitor compound having the structure (I), (IV) or (V) (or composition comprising such compound) effective to inhibit GSK3 activity in the subject. Other embodiments provided methods for treating a cell or ischemic injury or damage in a human or animal subject, comprising administering to the cell or to the human or animal subject an amount of a compound or composition of the invention effective to inhibit GSK3 activity in the cell or subject. Preferably, the subject will be a human or non-human animal subject. Inhibition of GSK3 activity includes detectable suppression of GSK3 activity either as compared to a control or as compared to expected GSK3 activity.

Effective amounts of the compounds of the invention generally include any amount sufficient to detectably inhibit GSK3 activity by any of the assays described herein, by other GSK3 kinase activity assays known to those having ordinary skill in the art or by detecting an inhibition or alleviation of ischemic damage or injury in a subject afflicted with an ischemia causing disorder, such as stroke.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be from about 0.1 mg/kg/day to about 100 mg/kg/day, preferably from about 1 mg/kg/day to about 20 mg/kg/day, and most preferably from about 2 mg/kg/day to about 10 mg/kg/day of a GSK3 inhibitor compound of the present invention, which may be administered in one or multiple doses.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

In another aspect, the methods and GSK3 inhibitor compounds of the invention may by used alone, or may be used in combination with one or more additional agents for the treatment of ischemic stroke, as are commonly employed in stroke therapy. Useful additional agents include, for example, thrombolytic and/or fibrinolytic agents that help reestablish cerebral circulation by dissolving (lysing) the clots which obstruct blood flow, neuroprotective agents that work to minimize the effects of the ischemic cascade, anticoagulants and antiplatelet agents. Representative thrombolytic agents include, for example, alteplase (tissue plasminogen activator (t-PA)), an enzyme found naturally in the body which converts, or activates, plasminogen into the enzyme plasmin to dissolve a blood clot; anistreplase; reteplase; urokinase; and streptokinase. Representative neuroprotective agents include, for example, glutamate antagonists that interfere with the progression of glutamate into the neurons, calcium antagonists that block the intracellular build-up of calcium through electrically-operated channels, opiate antagonists that interfere with the ischemic cascade by working on the opiate receptors which are overstimulated by the chemical cascade that occurs during cellular death, GABA-A agonists such as Clomethiazole (Astra) that activate the GABA-A receptor and thereby counteract the electrical activity of certain glutamate receptors, calpain inhibitors, NMDA receptor antagonists such as C-101,606 (Pfizer), K⁺ channel modulators such as BMS-204352 (Bristol-Myers Squibb), PDH kinase inhibitors, and antioxidants that scavenge free radicals generated by the ischemic cascade. In addition, the methods and compounds of the invention may be used in combination with oxygenated fluorocarbon nutrient emulsion (OFNE) therapy and/or neuroperfusion in which oxygen-rich blood is rerouted through the brain to reduce damage from an ischemic stroke. Representative anticoagulants include, for example, heparin, warfarin, dalteparin, danaparoid, enoxaparin, tinzaparin, 4-hydroxycoumarin, dicumarol, phenprocoumon, acenocoumarol, anisindone, lepirudin and indane-1,3-dione. Representative antiplatelet agents include, for example, aspirin, clopidogrel, ticlopidine, abciximab, eptifibatide, tirofiban and dipyridamole. When additional active agents are used in combination with the compounds of the present invention, the additional active agents may generally be employed in therapeutic amounts as indicated in the PHYSICIANS' DESK REFERENCE (PDR) 53^{rd} Edition (1999), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

The foregoing and other aspects of the invention may be better understood in connection with the following representative example.

### EXAMPLE

### Screening for GSK3 Inhibitory Activity Using a Cell-Based Glycogen Synthase Assay

CHO-HIRC cells are maintained in 10 cm tissue culture plates in Ham's F12 medium/10% dialyzed fetal bovine serum. Cells from a confluent 10 cm plate are harvested and divided into the 6 wells of a 6-well tissue culture plate to a final volume of 2 ml of medium. The cells are left to grow at 37° C. for 24 hours. The cells are then washed three times in Ham's F12 medium containing no fetal bovine serum, and finally the cells are left for a further 24 hours at 37° C. in 2 ml of the serum-free medium.

At the end of this time, 20 μl of compound dissolved in DMSO is added to each well and incubated at 37° C. After 20 minutes the medium is removed and the cells are washed once in PBS at room temperature and then rapidly frozen in the plates in liquid nitrogen. Cells are then thawed on ice in the presence of 140 μl of lysis buffer (50 mM Tris pH 7.8; 1 mM EDTA, 100 mM NaF, 25 μg/ml leupeptin, 1 mM DTT, 1 mM PMSF) per well. Cells are scraped from the plates and frozen in Eppendorf tubes on dry ice. Lysates are then thawed and refrozen on dry ice.

After rethawing, lysates are spun at 14,000 g for 15 minutes. The supernatants are then removed and stored on ice. Each supernatant (45 μl) is added to 45 μl of reaction buffer (65 mM Tris pH 7.8; 26 mM EDTA, 32.5 mM KF, 9.3 mM UDP-glucose; 11 mg/ml glycogen; 500 nCi/ml ¹⁴C-UDP-glucose) and a further 45 μl is added to 45 μl reaction buffer/20 mM glucose-6-phosphate. Reactions are incubated at 30° C. for 30 minutes and then spotted onto a 2 cm square 31ET chromatograph paper (Whatman). Filter papers are washed twice for 20 minutes in 66% ethanol, rinsed briefly in acetone and dried for 1 hour at room temperature.

Filters are added to 5 ml of liquid scintillant and counted in a liquid scintillation counter. The percentage of the total glycogen synthase that is active in any lysate is expressed as 100× (cpm minus glucose-6-phosphate)/(cpm plus glucose-6-phosphate). Such values are determined in duplicate for 5 different concentrations of compound and for DMSO alone, and the values are then plotted against the logarithm of the concentration. The concentration of compound which stimulates glycogen synthase activity to 50% of the maximal level is determined by fitting a sigmoidal curve to the plotted data. The maximal level is defined as that level to which glycogen synthase activity tends asymtotically as the concentration of test compound increases substantially beyond the EC₅₀. Representative GSK3 inhibitor compounds useful in the practice of the invention are shown in the following Table 1:

TABLE 1
GSK3 Inhibitor EC₅₀ Values

| Compound | Structure | EC₅₀ (μM) |
|---|---|---|
| D | 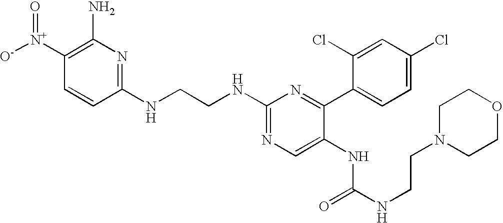 | 3 |
| E | 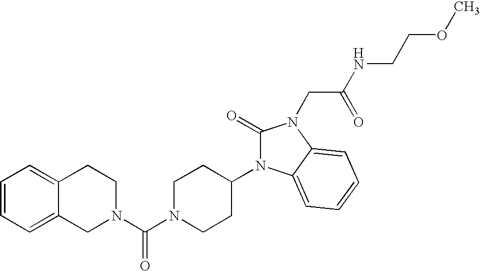 | |
| F | 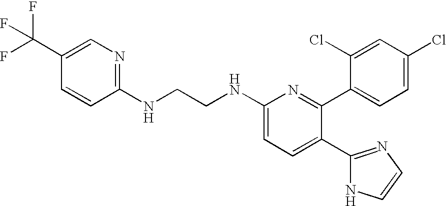 | |
| G | 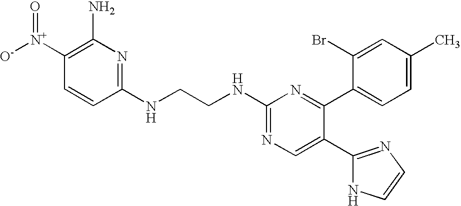 | 0.053 |
| H | 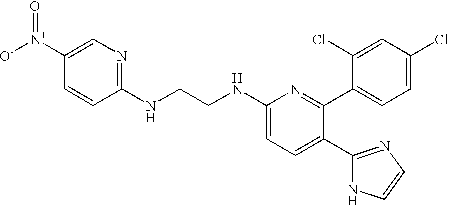 | 0.121 |
| I | 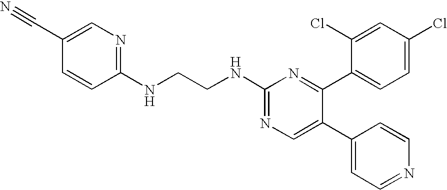 | 0.184 |
| J | 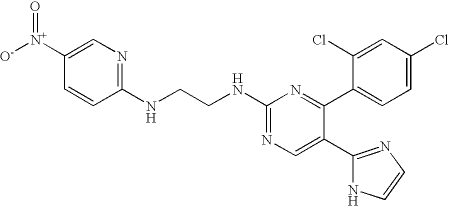 | 0.3747 |
| K | 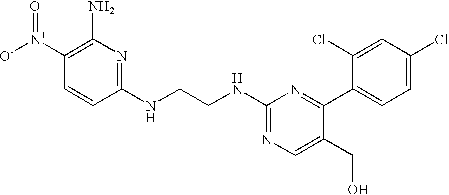 | 0.1471 |
| L | 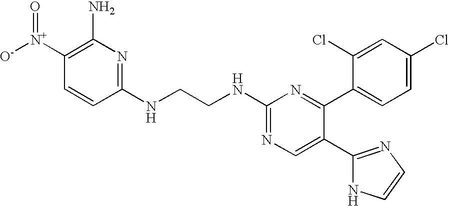 | 0.2566 |
| M | 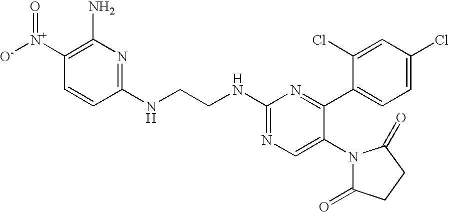 | 0.085 |
| N | 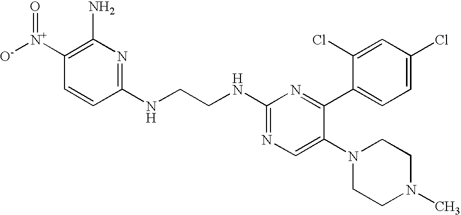 | 0.0534 |
| O | 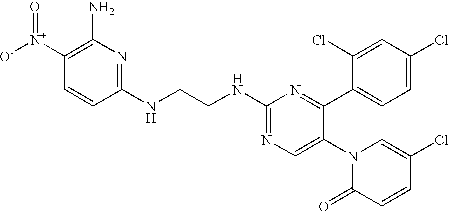 | 0.1325 |
| P | 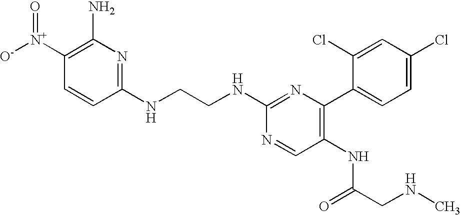 | 0.701 |
| Q | 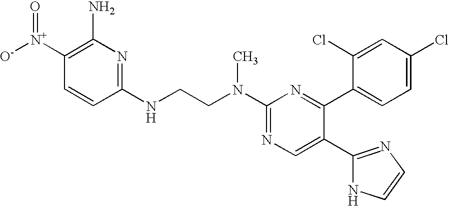 | 0.017 |
| R | 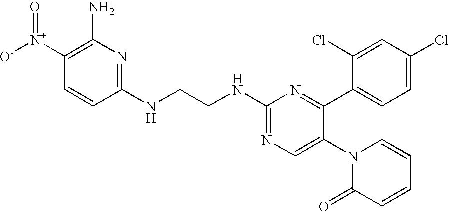 | 0.014 |
| S | 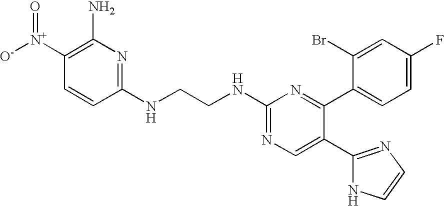 | 0.22 |
| T | 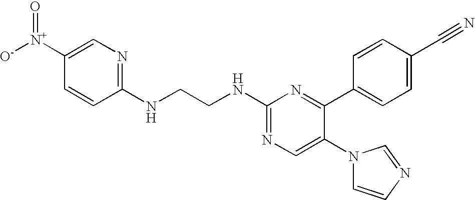 | 1.2828 |
| U | 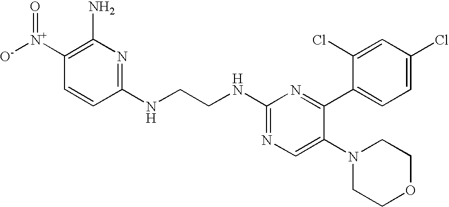 | 0.0735 |
| V | 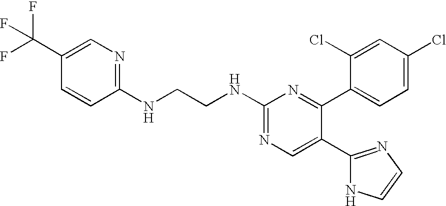 | 1.2378 |
| W | 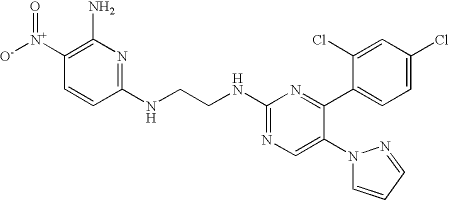 | 0.0998 |
| X | 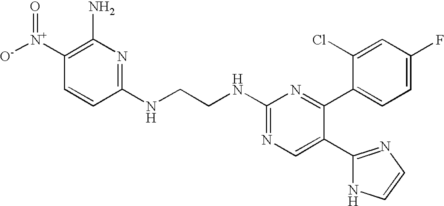 | 0.4242 |
| Y | 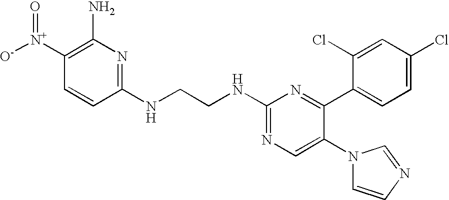 | 0.075 |

### EXAMPLE 2

### GSK3 Inhibitor Protection of Rat Hippocampi Cells From Glutamate Induced Damage

Hippocampi were dissected from embryonic day 18-19 rats. The tissue was collected in Hibernate™ media (Gibco BRL) and minced into 1 mm pieces. The tissue was dissociated using the Papain Dissociation System (Worthington Biochemical Corporation). Following isolation the cells were suspended in serum-free media composed of Neurobasal™ (Gibco BRL), 2% B27 supplement (Gibco BRL), L-glutamine and antibiotics. Cells were plated in 12 well plates tissue culture dishes coated with poly-L-lysine at a concentration of 7.5×10⁴ cells per well. Following 10-14 days at 37° C. in 5% CO₂, the GSK3 inhibitor compounds set forth below were added. Four to eight hours following compound addition, the conditioned media was removed from the cells and the cultures were stored at 37° C. The cultures were rinsed twice with HEPES buffered balanced salt solution (HBSS) containing 10 μM glycine (Grabb and Choi, 1999). The cultures were then either exposed for 5-minutes at room temperature to 200 μM glutamic acid in the same HBSS, or not exposed to glutamic acid as a control. Following exposure, the cultures were rinsed three times with the buffer and then returned to their original conditioned media containing the GSK3 inhibitor compounds. Twenty to 24 hours following glutamic acid exposure, the cultures were rinsed in HBSS and exposed for 10 minutes to Trypan Blue. This dye is taken up by dead cells. The cultures were rinsed then fixed for 30 minutes in 4% paraformaldehyde. The number of live and dead (blue) large neurons are counted (at least 200 cells from each well) by phase contrast microscopy and photographed, and the percentage of surviving cells was determined. The foregoing procedure was followed using no GSK3 inhibitor compound (A), dimethylsulfoxide (DMSO, B), and IGF1 as controls, and with the GSK3 inhibitor compounds D-Y (Table 1). The results are shown in the following Table 2 as the average of 1 to 4 replications:

TABLE 2
GSK3 Inhibitor Enhanced Cell Survival

| | Cells Surviving (%) | |
|---|---|---|
| | Without Glutamate | |
| Compound | (control) | With Glutamate |
| NA | 95 | 48 |
| DMSO | 94 | 46 |
| IGF1 | 93 | 51 |
| D | 96 | 60 |
| E | 88 | 73 |
| F | 96 | 51 |
| G | 94 | 65 |
| H | 94 | 67 |
| I | 94 | 56 |
| J | 95 | 57 |
| K | 86 | 73 |
| L | 90 | 59 |
| M | 87 | 61 |
| N | 91 | 67 |
| O | 94 | 61 |
| P | 91 | 55 |
| Q | 95 | 67 |
| R | 92 | 61 |
| S | 95 | 50 |
| T | 95 | 50 |
| U | 93 | 62 |
| V | 94 | 49 |
| W | 94 | 61 |
| X | 93 | 50 |
| Y | 94 | 61 |

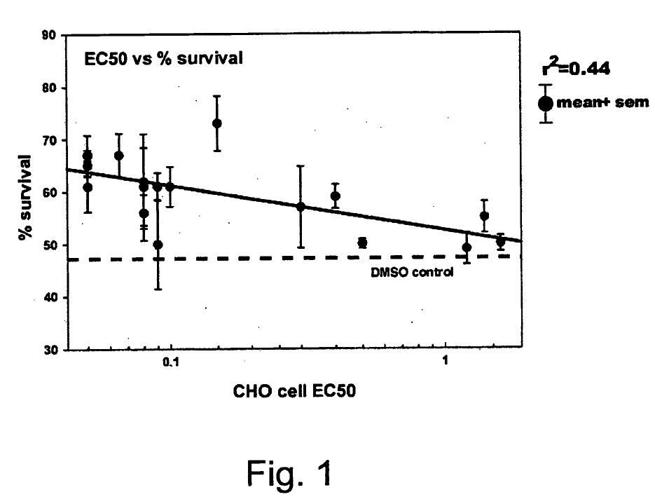

A plot of the percentage survival versus CHO cell EC₅₀ values (Table 1) is shown in FIG. 1, which reflects a direct relationship between EC₅₀.values of the GSK3 inhibitor compounds and cell survival.

### EXAMPLE 3

### Partition Coefficients of Representative GSK3 Inhibitors

logP is the logarithm of the partition coefficient of the neutral form of a compound between octanol and water. It is a physico-chemical parameter that has a correlation with absorption of small molecules into physiological membranes. The logP of representative GSK3 inhibitor compounds D-Y (Table 1) was determined using the logP prediction program PrologP 5.1 (CompuDrug International, Inc. 705 Grandview Drive, South San Francisco, Calif. 94080 USA). PrologP is an add-on module of the PALLAS system, which serves as a frame of modules predicting physico-chemical features of compounds. The PrologP 5.1 computer program predicts the logP values based on the structural formulas of the compounds. The program uses three different systems for the prediction. These systems disintegrate the compound to fragments, and express the logP value as a superposition of the corresponding fragment constants (Broto et al., Eur. J. Med. Chem.—Chim. Ter. 19:71 (1984); Ghose et al., J. Computat. Chem. 7:565 (1986)). The program uses about 150 atomic fragments (Viswanadhn et al, J. Chem. Inf. Comput. Sci., 29:163-172 (1989)). The predicted results of these methods are combined in order to minimize the error of the estimation. The logP values for representative GSK3 inhibitor compounds D-Y (Table 1) are shown in the following Table 3:

TABLE 3
GSK3 Inhibitor Partition Coefficients

| | Partition |
|---|---|
| Compound | Coefficients) |
| D | 5.62 |
| E | 3.23 |
| F | 6.03 |
| G | 4.41 |
| H | 4.94 |
| I | 4.4 |
| J | 4.29 |
| K | 3.67 |
| L | 4.53 |
| M | 4.04 |
| N | 5.76 |
| O | 6.06 |
| P | 3.54 |
| Q | 4.63 |
| R | 6 |
| S | 4.06 |
| T | 3.05 |
| U | 5.2 |
| V | 5.38 |
| W | 5.49 |
| X | 3.96 |
| Y | 5.03 |

### EXAMPLE 4

### GSK3 Inhibitor Blood Brain Barrier Penetration

GSK3 inhibitor blood brain barrier penetration may generally be measured by administering a GSK3 inhibitor compound formulated in 15% captisol/20 mM sodium citrate to a final dose of 10 mg/kg intravenously through a jugular catheter into 250 g CD rats. At 150 minutes post administration brains from the rats were isolated and fast frozen. GSK3 inhibitor plasma levels in the isolated material were established using standard techniques. The results for compounds G and I (Table 1) are shown in the following Table 4:

TABLE 4
GSK3 Inhibitor Brain Concentrations

mean plasma ng/ml
drug levels

| Time (min) | Cmpd. G | Cmpd. I |
|---|---|---|
| 0 | <20 | <20 |
| 7 | 4046 | 1191 |
| 20 | 3198 | 1160 |
| 40 | 1329 | 614 |
| 70 | 796 | 440 |
| 100 | 414 | 358 |
| 150 | 113 | 106 |
| Brain (at end of | <LOQ | <LOQ |
| study) | | |
| Parameter summary: | | |
| AUC | 191 | 8.7 |
| (μg*min/ml) | | |
| Cmax (μg/ml) | 4.6 | 1.2 |
| Tmax (min) | ˜0 | ˜0 |
| C_{L} ([ml/min]/kg} | 52 | 114 |
| Vss (L/kg) | 2.3 | 6.8 |
| t_{1/2} (min) | 31 | 42 |

### EXAMPLE 5

### Transient (90 min.) Middle Cerebral Artery Occlusion (MCAO) in SD Rats

1. Transient focal cerebral ischemia: A rodent model of transient middle cerebral artery occlusion (MCA) occlusion was used to determine the effect of a representative GSK3 inhibitor compound of the invention, 6-[(2-{[6-(2,4-dichlorophenyl)-5-imidazolyl-2-pyridyl]amino}ethyl)amino]pyridine-3-carbonitrile (CT 99025) in the inhibition of ischemic damage due to stroke. Ischemia was induced using an occluding intraluminal suture model (M. A. Yenari et al., “Time-course and treatment response with SNX-111, an N-type calcium channel blocker, in a rodent model of focal cerebral ischemia using diffusion-weighted MRI,” Brain Res 739:3645 (1996)). Animals were anesthetized with 3% halothane (1-2% maintenance) via face mask. Depth of anesthesia was monitored every 15 minutes by checking toe pinch, and the maintenance halothane levels were adjusted accordingly. A lateral neck dissection was performed and the common, internal and external carotid arteries are identified. A 3-0 monofilament suture with the tip rounded by a flame was inserted into the common carotid artery (CCA) and advanced 18-20 mm into the internal carotid artery (ICA) under direct observation. The occluding suture was kept in place for 90 minutes, then removed to allow for reperfusion. During the surgical procedure, the following parameters were monitored and maintained in the normal physiological range: mean arterial blood pressure (MABP, measured via a femoral artery catheter), respiratory rate, rectal temperature, heart rate, arterial blood gases, blood glucose, and hematocrit. The animals were allowed to recover, then transported to the intensive care unit at the animal facility for post-operative monitoring. At the completion of the experiment (3 days), the animals were euthanized by a halothane or barbiturate overdose, then transcardially perfused with 3% paraformaldehyde. The brains were removed then fixed in a similar solution with 20% sucrose at 4° C. for 24-48 hours. Brain sections were prepared for histological analysis.

2. Treatment

Immediately after the start of the artery occlusion, intravenous bolus of vehicle (15% captisol) or CT 99025 at 25 mg/kg was given to the rats. At the end of the occlusion (90 minutes), rats received a subcutaneous injection of vehicle or CT 99025 at 50 mg/kg. Another subcutaneous injection was given 8 hours later and every 12 hours thereafter for the next 60 hours.

3. Histological methods: Standard immunohistochemical methods were be used. To delineate regions of infarction, brain sections were stained with cresyl violet. Regions without staining were delineated using a computer assisted image analysis program (MCID, St. Catherine's, Ottawa), and expressed as the percent injury of the ipsilateral hemisphere. Adjacent sections are saved at −70° C. for future histochemical stains.

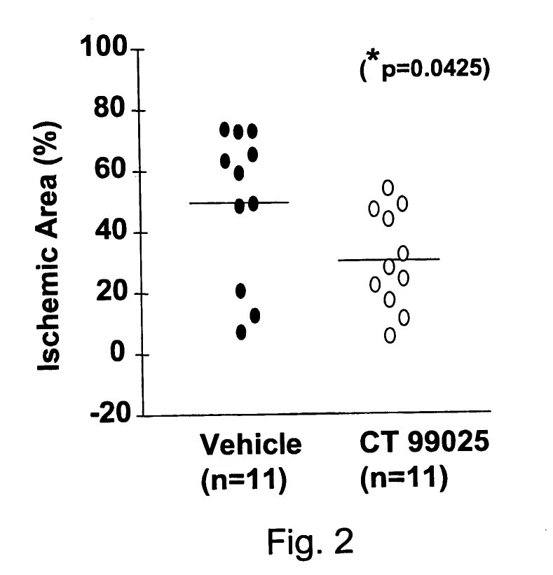

As shown in FIG. 2, the ischemic area in the rats in the group treated with the GSK3 inhibitor, 6-[(2-{[6-(2,4-dichlorophenyl)-5-imidazolyl-2-pyridyl]amino}ethyl)-amino]pyridine-3-carbonitrile (shown in FIG. 2 as CT 99025), is substantially reduced over that of the control group (vehicle).

While the preferred embodiment of the invention has been illustrated and described, it will be appreciated that various changes can be made therein without departing from the spirit and scope of the invention.

1. A method for the treatment of a human or animal subject comprising administrating to the subject within 24 hours of the onset of an ischemic stroke event an amount of a glycogen synthase kinase 3 (GSK3) inhibitor effective to reduce or prevent ischemic injury in the subject.

2. A method of claim 1 wherein the GSK3 inhibitor is administered to the subject within 8 hours of the onset of the ischemic stroke event.

3. A method of claim 1 wherein the GSK3 inhibitor is administered to the subject within 2 hours of the onset of the ischemic stroke event.

4. A method of claim 1 wherein the GSK3 inhibitor is administered to the subject intermittently or continuously for at least 24 hours.

5. A method of claim 1 wherein the GSK3 inhibitor has a molecular weight below about 800.

6. A method of claim 1 wherein the GSK3 inhibitor has a molecular weight below about 500.

7. A method of claim 1 wherein the GSK3 inhibitor has a molecular weight below about 400.

8. A method of claim 1 wherein the GSK3 inhibitor has a log P in the range of about 0 to 8.

9. A method of claim 1 wherein the GSK3 inhibitor has a log P in the range of about 1 to 6.

10. A method of claim 1 wherein the GSK3 inhibitor has a log P in the range of about 2 to 5.

11. A method of claim 1 wherein the GSK3 inhibitor is administered to the subject in combination with at least one additional agent for the treatment of ischemic stroke.

12. A method of claim 11 wherein the additional agent for the treatment of ischemic stroke is selected from the group consisting of thrombolytic agents, fibrinolytic agents, neuroprotective agents, anticoagulants and antiplatelet agents.

13. A method of claim 11 wherein the additional agent for the treatment of ischemic stroke is a thrombolytic agent selected from the group consisting of alteplase, anistreplase, reteplase, urokinase and streptokinase.

14. A method of claim 11 wherein the additional agent for the treatment of ischemic stroke is a neuroprotective agent selected from the group consisting of caspase inhibitors, glutamate antagonists, calcium antagonists, opiate antagonists, GABA-A agonists, calpain inhibitors, NMDA receptor antagonists, K⁺ channel modulators, PDH kinase inhibitors, and antioxidants.

15. A method of claim 11 wherein the additional agent for the treatment of ischemic stroke is an anticoagulant selected from the group consisting of heparin, warfarin, dalteparin, danaparoid, enoxaparin, tinzaparin, 4-hydroxycoumarin, dicumarol, phenprocoumon, acenocoumarol, anisindone, lepirudin and indane-1,3-dione.

16. A method of claim 11 wherein the additional agent for the treatment of ischemic stroke is an antiplatelet agent selected from the group consisting of aspirin, clopidogrel, ticlopidine, abciximab, eptifibatide, tirofiban and dipyridamole.

17. A method for treating cerebrovascular ischemic disorders in a human or animal subject in need of such treatment comprising administering to said subject an amount of a glycogen synthase kinase 3 (GSK3) inhibitor effective to reduce or prevent ischemic injury in the subject in combination with at least one additional agent for the treatment of ischemic stroke.

18. A method of claim 17 wherein the glycogen synthase kinase 3 (GSK3) inhibitor is administered to the subject prior to administration of the at least one additional agent.

19. A method of claim 17 wherein the glycogen synthase kinase 3 (GSK3) inhibitor is administered to the subject concurrently with administration of the at least one additional agent.

20. A method of claim 17 wherein the glycogen synthase kinase 3 (GSK3) inhibitor is administered to the subject prior to and concurrently with administration of the at least one additional agent.

21. A method of claim 17 wherein the GSK3 inhibitor is administered to the subject within 24 hours of the onset of an ischemic stroke event in the subject.

22. A method of claim 17 wherein the GSK3 inhibitor is administered to the subject within 8 hours of the onset of an ischemic stroke event in the subject.

23. A method of claim 17 wherein the GSK3 inhibitor is administered to the subject within 2 hours of the onset of an ischemic stroke event in the subject.

24. A method of claim 17 wherein the GSK3 inhibitor is administered to the subject intermittently or continuously for at least 24 hours.

25. A method of claim 17 wherein the GSK3 inhibitor has a molecular weight below about 800.

26. A method of claim 17 wherein the GSK3 inhibitor has a molecular weight below about 500.

27. A method of claim 17 wherein the GSK3 inhibitor has a molecular weight below about 400.

28. A method of claim 17 wherein the GSK3 inhibitor has a log P in the range of about 0 to 8.

29. A method of claim 17 wherein the GSK3 inhibitor has a log P in the range of about 1 to 6.

30. A method of claim 17 wherein the GSK3 inhibitor has a log P in the range of about 2 to 5.

31. A method of claim 17 wherein the additional agent for the treatment of ischemic stroke is selected from the group consisting of thrombolytic agents, fibrinolytic agents, neuroprotective agents, anticoagulants and antiplatelet agents.

32. A method of claim 31 wherein the additional agent for the treatment of ischemic stroke is a thrombolytic agent selected from the group consisting of alteplase, anistreplase, reteplase, urokinase and streptokinase.

33. A method of claim 31 wherein the additional agent for the treatment of ischemic stroke is a neuroprotective agent selected from the group consisting of caspase inhibitors, glutamate antagonists, calcium antagonists, opiate antagonists, GABA-A agonists, calpain inhibitors, NMDA receptor antagonists, K⁺ channel modulators, PDH kinase inhibitors, and antioxidants.

34. A method of claim 31 wherein the additional agent for the treatment of ischemic stroke is an anticoagulant selected from the group consisting of heparin, warfarin, dalteparin, danaparoid, enoxaparin, tinzaparin, 4-hydroxycoumarin, dicumarol, phenprocoumon, acenocoumarol, anisindone, lepirudin and indane-1,3-dione.

35. A method of claim 31 wherein the additional agent for the treatment of ischemic stroke is an antiplatelet agent selected from the group consisting of aspirin, clopidogrel, ticlopidine, abciximab, eptifibatide, tirofiban and dipyridamole.

36. A composition comprising a GSK3 inhibitor and at least one additional agent for the treatment of ischemic stroke.

37. A composition of claim 36 wherein the GSK3 inhibitor has a molecular weight below about 800.

38. A composition of claim 36 wherein the GSK3 inhibitor has a molecular weight below about 500.

39. A composition of claim 36 wherein the GSK3 inhibitor has a molecular weight below about 400.

40. A composition of claim 36 wherein the GSK3 inhibitor has a log P in the range of about 0 to 8.

41. A composition of claim 36 wherein the GSK3 inhibitor has a log P in the range of about 1 to 6.

42. A composition of claim 36 wherein the GSK3 inhibitor has a log P in the range of about 2 to 5.

43. A composition of claim 36 wherein the additional agent for the treatment of ischemic stroke is selected from the group consisting of thrombolytic agents, fibrinolytic agents, neuroprotective agents, anticoagulants and antiplatelet agents.

44. A composition of claim 36 wherein the additional agent for the treatment of ischemic stroke is a thrombolytic agent selected from the group consisting of alteplase, anistreplase, reteplase, urokinase and streptokinase.

45. A composition of claim 44 wherein the additional agent for the treatment of ischemic stroke is a neuroprotective agent selected from the group consisting of caspase inhibitors, glutamate antagonists, calcium antagonists, opiate antagonists, GABA-A agonists, calpain inhibitors, NMDA receptor antagonists, K⁺ channel modulators, PDH kinase inhibitors, and antioxidants.

46. A composition of claim 44 wherein the additional agent for the treatment of ischemic stroke is an anticoagulant selected from the group consisting of heparin, warfarin, dalteparin, danaparoid, enoxaparin, tinzaparin, 4-hydroxycoumarin, dicumarol, phenprocoumon, acenocoumarol, anisindone, lepirudin and indane-1,3-dione.

47. A composition of claim 44 wherein the additional agent for the treatment of ischemic stroke is an antiplatelet agent selected from the group consisting of aspirin, clopidogrel, ticlopidine, abciximab, eptifibatide, tirofiban and dipyridamole.

48. The use of a GSK3 inhibitor in the manufacture of a medicament for the treatment of cerebrovascular ischemic disorders.

49. A method of claim 1 wherein the GSK3 inhibitor is administered by a route selected from the group consisting of oral, subcutaneous, transdermal, transmucosal, iontophoretic, intracerebral, intravenous, intrarterial, intramuscular, intraperitoneal, intranasal, intrathecal, subdural, and rectal routes of administration.

50. A method of claim 49 wherein the GSK3 inhibitor is administered systemically.

51. A method of claim 49 wherein the GSK3 inhibitor is administered intracerebrally.

52. A method of claim 49 wherein the GSK3 inhibitor is administered intrathecally.

53. A method of claim 17 wherein the GSK3 inhibitor is administered by a route selected from the group consisting of oral, subcutaneous, transdermal, transmucosal, iontophoretic, intracerebral, intravenous, intraarterial, intramuscular, intraperitoneal, intranasal, intrathecal, subdural, and rectal routes of administration.

54. A method of claim 53 wherein the GSK3 inhibitor is administered systemically.

55. A method of claim 53 wherein the GSK3 inhibitor is administered intracerebrally.

56. A method of claim 53 wherein the GSK3 inhibitor is administered intrathecally.
